(19) United States
(12) Patent Application Publication
(10) Pub. No.: US 2025/0285223 A1
(43) Pub. Date: Sep. 11, 2025

(54) METHODS AND DEVICES FOR ACQUIRING SUPER-RESOLUTION IMAGING WITH MODULATION

(71) Applicant: The University of Chicago, Chicago, IL (US)

(72) Inventor: Chien-Min Kao, Wilmette, IL (US)

(21) Appl. No.: 19/215,512

(22) Filed: May 22, 2025

Related U.S. Application Data
(63) Continuation of application No. PCT/US2023/083127, filed on Dec. 8, 2023.
(60) Provisional application No. 63/431,417, filed on Dec. 9, 2022.

(51) Int. Cl. G06T 3/4076

(52) U.S. Cl. CPC G06T 3/4076

(57) ABSTRACT

The present disclosure describes various methods, systems, and storage medium for acquiring super-resolution images with modulation. An apparatus includes a periodic pattern and a detector. The periodic pattern is configured to move to modify an original image field before the original image field propagates onto the detector of an imaging system, the original image field originating from a subject and comprising information of the subject, and the detector is configured to detect the modified original image field to acquire a plurality of detected images. The apparatus also includes a memory storing instructions and a processor in communication with the memory. When the processor executes the instructions, the processor is configured to cause the apparatus to reconstruct a super-resolution image based on the plurality of detected images, wherein an imaging resolution of the super-resolution image is better than an intrinsic resolution of the detector.

### RELATED APPLICATION

This application is a continuation application of PCT International Patent Application No. PCT/US2023/083127, filed on Dec. 8, 2023, which is herein incorporated by reference in its entirety. PCT International Patent Application No. PCT/US2023/083127 is based on and claims the benefit of priority to U.S. Provisional Application No. 63/431,417, filed on Dec. 9, 2022, which is herein incorporated by reference in its entirety.

### FIELD OF THE INVENTION

This disclosure relates to a method and system for acquiring a super-resolution image of a subject with modulating absorption or sensitivity patterns.

### BACKGROUND OF THE INVENTION

Many biomedical imaging modalities are limited by their intrinsic resolutions, impeding effective diagnosis and/or treatment of certain diseases with high sensitivity and high specificity. For example, a resolution of some clinical positron emission tomography (PET) may be around 4 millimeter (mm), which may be insufficient for detecting early stage lesions and imaging specific organs or regions such as the head-and-neck, pancreas, and/or brain, that have important small structures or delicate anatomy.

The present disclosure describes various embodiments for acquiring a super-resolution image of a subject with modulating absorption or sensitivity patterns. The obtained resolution of various embodiments in the present disclosure is beyond its intrinsic instrumentation limit, providing an relatively inexpensive and easy-to-use add-on device for some pre-exiting imaging systems and/or developing high-end biomedical systems, and thus, improving biomedical imaging field.

### SUMMARY OF THE INVENTION

In view of this, embodiments of the present disclosure are expected to provide a method, apparatus, and a storage medium for acquiring a super-resolution image of a subject with modulating absorption or sensitivity patterns.

In one embodiment, the present disclosure describes an apparatus for acquiring a super-resolution image of a subject. The apparatus includes a periodic pattern and a detector. The periodic pattern is configured to move to modify an original image field before the original image field propagates onto the detector of an imaging system, the original image field originating from a subject and comprising information of the subject, and the detector is configured to detect the modified original image field to acquire a plurality of detected images. The apparatus also includes a memory storing instructions and a processor in communication with the memory. When the processor executes the instructions, the processor is configured to cause the apparatus to reconstruct a super-resolution image based on the plurality of detected images, wherein an imaging resolution of the super-resolution image is better than an intrinsic resolution of the detector. In some implementations, the detector may include a pixelated detector, and/or may be a physical detector or a virtual detector. In some implementations, the periodic pattern may be configured to move by either physically moving the periodic pattern or being electronically simulated (e.g., changing sensitivity pattern electronically). For a non-limiting example, the subject may include a biological sample.

In another embodiment, the present disclosure describes a method for acquiring a super-resolution image of a subject. The method includes moving a periodic pattern to modify an original image field before the original image field propagates onto a detector of an imaging system, the original image field originating from a subject and comprising information of the subject; detecting the modified original image field with the detector to acquire a plurality of detected images; and reconstructing a super-resolution image based on the plurality of detected images, wherein an imaging resolution of the super-resolution image is better than an intrinsic resolution of the detector.

In another embodiment, the present disclosure describes an apparatus for acquiring a super-resolution image of a subject, the apparatus comprising: a detector of an image system, wherein: a modulation of the detector is configured to cause modulation of radiation beams, the radiation beams originating from a subject and comprising information of the subject, and the detector is configured to receive the radiation beam to acquire a plurality of detected images; and a memory storing instructions and a processor in communication with the memory, wherein, when the processor executes the instructions, the processor is configured to cause the apparatus to reconstruct a super-resolution image based on the plurality of detected images, wherein an imaging resolution of the super-resolution image is better than an intrinsic resolution of the detector.

In another embodiment, the present disclosure describes a method for acquiring a super-resolution image of a subject, the method comprising: modulating a detector of an imaging system to cause modulation of radiation beams, the radiation beams originating from a subject and comprising information of the subject; detecting the radiation beams with the detector to acquire a plurality of detected images; and reconstructing a super-resolution image based on the plurality of detected images, wherein an imaging resolution of the super-resolution image is better than an intrinsic resolution of the detector.

In some other embodiments, a device for acquiring a super-resolution image of a subject may include a memory storing instructions and a processing circuitry in communication with the memory. When the processing circuitry executes the instructions, the processing circuitry is configured to carry out any single step, any combinations of some steps, or all steps of the above described methods.

In some other embodiments, a system for acquiring a super-resolution image of a subject may include portions or all, or a combination of portions or all of the above described apparatus or device, and the system is configured to carry out any single step, any combinations of some steps, or all steps of the above described methods.

A non-transitory computer program product comprising a computer-readable program medium code stored thereupon, the computer-readable program medium code, when executed by a processor, causing the processor to perform a portion or all of the above methods.

The above and other aspects and their implementations are described in greater detail in the drawings, the descriptions, and the claims.

### BRIEF DESCRIPTION OF THE DRAWINGS

The system, device, product, and/or method described in the present disclosure may be better understood with reference to the following drawings and description of non-limiting and non-exhaustive embodiments. The components in the drawings are not necessarily to scale, and reference is made to the following drawings, in which:

FIG. 1 is a schematic diagram of an embodiment of an apparatus disclosed in the present disclosure;

FIG. 2 is a schematic diagram of another embodiment of an apparatus disclosed in the present disclosure;

FIG. 3 illustrates a schematic diagram of a classical computer system;

FIG. 4 is a flow diagram of an embodiment disclosed in the present disclosure;

FIG. 5 is a flow diagram of another embodiment disclosed in the present disclosure;

FIG. 6 is a schematic diagram of a detector of an imaging system disclosed in the present disclosure;

FIG. 7 is charts of computer simulated data for various embodiments in the present disclosure;

FIG. 8 is charts of computer simulated data at 43 different positions for various embodiments in the present disclosure;

FIG. 9 is charts of estimated spectra at 43 different positions for various embodiments in the present disclosure;

FIG. 10 is charts of images for various positions for various embodiments in the present disclosure;

FIG. 11 is charts of spectra for various positions for various embodiments in the present disclosure;

FIG. 12 shows examples of reconstructing process for various embodiments in the present disclosure;

FIG. 13 shows a schematic diagram for super resolution in position emission tomography (PET);

FIG. 14 shows a chart for super resolution imaging with undersampled data. In the (k, n)-space, k, n∈ [symbol] , points on the line [symbol] (m)={(k, n):kp+n=m}, p∈ [symbol] ⁺, contribute F(ν−mνₛ) in Eq. (15) if they are also in Γ={(k, n):|k|≤K, |n|≤N}; that is, if they are points on [symbol] _{Γ}(m)= [symbol] (m)∩Γ. The case with p=3, K=4, and N=3 is shown;

FIG. 15 shows another chart for super resolution imaging with undersampled data. In the (k, n)-space, k, n∈ [symbol] , points on [symbol] _{Γ}′(m)= [symbol] ′(m)∩Γ contribute F(ν−mνₛ) in Eq. (17) where [symbol] ′(m)={(n, k):k+nq=m}. The case with q=2, K=4, and N=3 is shown;

FIG. 16 shows another chart for super resolution imaging with undersampled data. For even p and m=p/2, H(ν+pνₛ/2) and H(ν−pνₛ/2) are the two nearest terms to ν=0 in Eq. (16) when extended to sum over [symbol] (m). If ν₀>2σ, there exists a gap interval I_{g} not covered by them and cₗₘ(ν)=0 for any ν∈I_{g};

FIG. 17 shows another chart for super resolution imaging with undersampled data. (a) aₗₘ(γ)=0 in the shaded areas defined by rk+ [symbol] >Γ₋(γ) or rk+ [symbol] <−Γ₊(γ). The boundary line L₊ passes through the point (0, Γ₋(γ) and L₋ through (0,−Γ₊(γ)). A horizontal/vertical line present in these shaded areas gives a row/column of zeros in [symbol] (γ). (b) Line L₊^{min} is defined by rₘᵢₙ⁺k+ [symbol] =Γ₋(γ), rₘᵢₙ⁺=1−Γ₋(γ)/K** so that it passes through (−K**, +K**). On the other hand, L₊^{max} is defined by rₘₐₓ⁺k+ [symbol] =Γ₋(γ), rₘₐₓ⁺=1+Γ₋(γ)/K** so that it passes through (+K**, −K**). Lines Imin and Imax are similarly defined are defined, yielding r₋^{min}=1−Γ₊(γ)/K** and r₋^{max}=1+Γ+(γ)/K**;

FIG. 18 shows reconstruction images with different sampling modes and iterations. From the top to bottom rows are normal, p0, p1, p2 mode. From left to right columns, the number of iterations is 50, 100, 250, and 500;

FIG. 19 shows spectrum of phantom and four sampling mode in 500 iterations. The inner black circle is theoretical cutoff frequency and the outer one is double of it;

FIG. 20 shows reconstruction images of four different sampling modes at the same scan time relative to normal sampling mode; and

FIG. 21 shows contrast-recovery-coefficient (CRC) versus background noise (BN) curves at different rod diameters from 0.9 mm to 2.4 mm (top left to bottom right).

### DETAILED DESCRIPTION

The description and accompanying drawings above provide specific example embodiments and implementations. Drawings containing device structure and composition, for example, are not necessarily drawn to scale unless specifically indicated. Subject matter may, however, be embodied in a variety of different forms and, therefore, covered or claimed subject matter is intended to be construed as not being limited to any example embodiments set forth herein. A reasonably broad scope for claimed or covered subject matter is intended. Among other things, for example, subject matter may be embodied as methods, devices, components, or systems. Accordingly, embodiments may, for example, take the form of hardware, software, firmware or any combination thereof.

Throughout the specification and claims, terms may have nuanced meanings suggested or implied in context beyond an explicitly stated meaning. Likewise, the phrase “in one embodiment/implementation” as used herein does not necessarily refer to the same embodiment and the phrase “in another embodiment/implementation” as used herein does not necessarily refer to a different embodiment. It is intended, for example, that claimed subject matter includes combinations of example embodiments in whole or in part.

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred/exemplary methods and materials are described herein.

In general, terminology may be understood at least in part from usage in context. For example, terms, such as “and”, “or”, or “and/or,” as used herein may include a variety of meanings that may depend at least in part on the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The embodiments of the present disclosure provide a method, an apparatus, and a non-transitory computer readable storage medium for acquiring a super-resolution image of a subject with modulating absorption or sensitivity patterns.

Many biomedical imaging modalities are limited by their intrinsic resolutions, impeding effective diagnosis and/or treatment of certain diseases with high sensitivity and high specificity. For example, a resolution of some clinical positron emission tomography (PET) may be around 4 millimeter (mm), which may be insufficient for detecting early stage lesions and imaging specific organs or regions such as the head-and-neck, pancreas, and/or brain, that have important small structures or delicate anatomy.

The present disclosure describes various embodiments for acquiring a super-resolution image of a subject with modulating absorption or sensitivity patterns. The present disclosure may include several exemplary imaging modalities, which merely serve as non-limiting examples, and the embodiments may be generally applicable to other imaging modalities (for example, PET, autoradiography, fluorescence, radioluminescence, computed tomography (CT), etc.). The obtained resolution of various embodiments in the present disclosure is beyond its intrinsic instrumentation limit, providing an relatively inexpensive and easy-to-use add-on device for some pre-exiting imaging systems and/or developing high-end biomedical systems, and thus, improving biomedical imaging field.

In some implementations, the technology may be related to a specific super-resolution technology for fluorescent microscopy. The technology allows one to overcome the intrinsic resolution limit of an instrument that employs pixelated sensors by introducing moving structured absorptive or sensitivity patterns to modulate the emission field, which may be of light photons, x-ray/gamma rays or charged particles, in a way that encodes its resolution content above the instrument's bandwidth to appear within and thereby become measurable even though it is mixed with the low-resolution content. To unmix them, several measurements are taken by moving the structured absorptive or sensitivity patterns to various positions, and a mathematical model relating the emission field and these measurements is solved. As a result, the spectrum of the emission field can be recovered to a frequency that is substantially above the instrument's bandwidth. For one non-limiting example, 1 micrometer (micron) resolution may be achieved from a camera that has a 12-micron intrinsic blur while employing a sensor consisting of a 24×24 array of 6-micro pixels.

In some implementations, in nuclear medicine, in vivo distribution of radio-labeled molecules or particles in tissues at a resolution of 100 microns-1 mm for animals and 4-10 mm for humans may be obtained. There is a need to understand the mechanisms that affect the distribution at the cellular level so that the in vivo imaging results may be better interpreted, either for basic research, disease diagnosis, or therapeutics development. As an example, it has been known that while nicotine is quickly cleared out some smoke cessation drugs stay in neurons for days or weeks. One hypothesis is that there exist previously unidentified intracellular organelles that contain nicotine receptors. In addition to binding the nicotine receptors at the cell plasma member, nicotine and its analogs can also bind these intracellular receptors. Since these organelles are acidic, a weak-base molecule may become charged and trapped inside the cell. If true, the uptake of many PET tracers may need to be reexamined and the results re-interpreted. In some implementations, autoradiography may be used to examine the distribution of radio-labeled molecules in a thin tissue slice at a resolution that can resolve tissue structures and still not cells; and currently available sensors may be demonstrated to achieve better resolution.

In some implementations, the described techniques may be applied to other imaging modalities. For example, an add-on rotating absorptive ring may be used for boosting the spatial resolution of an existing PET system. This add-on is much less expensive to develop than increasing the intrinsic resolution of the system. In some implementation, the described techniques may also be applied to achieve super high resolution CT. In some implementations, the described techniques may also be applied to improving optical cameras. For example, by introducing the moving structured absorption patterns in front of a lens, the resolution of a camera may be improved. Rather than increasing the resolution, the field of view (FOV) may be expanded. For example, if originally the camera has N pixels and allows a field of view of F to achieve a resolution of r=F/N. When the field of view is increased to mF, m>1, the area one pixel needs to cover also increases m fold, hence the resolution is degraded by m fold; and with the described technology, this loss in resolution can be recovered. This is like achieving the panorama mode without moving the camera. In some implementations, as the described techniques do not require lens, the technology may also be useful for developing lens-less cameras.

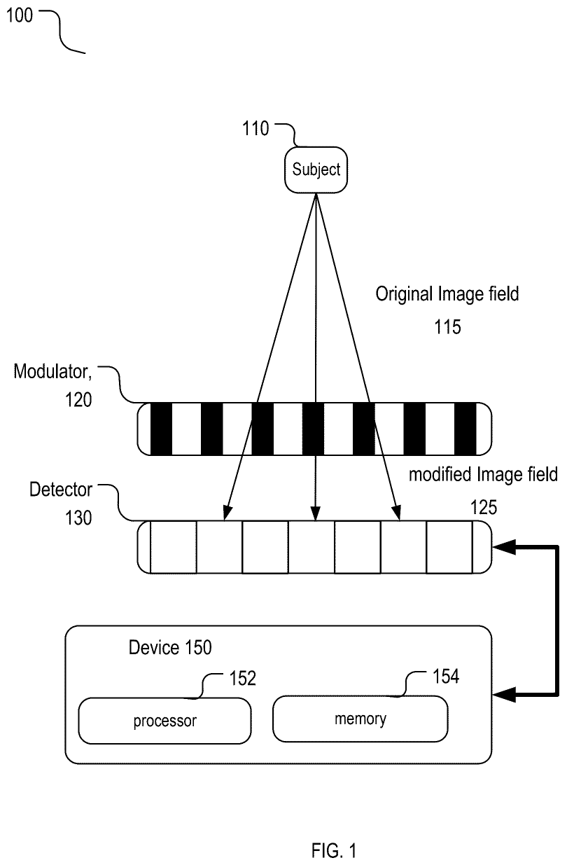

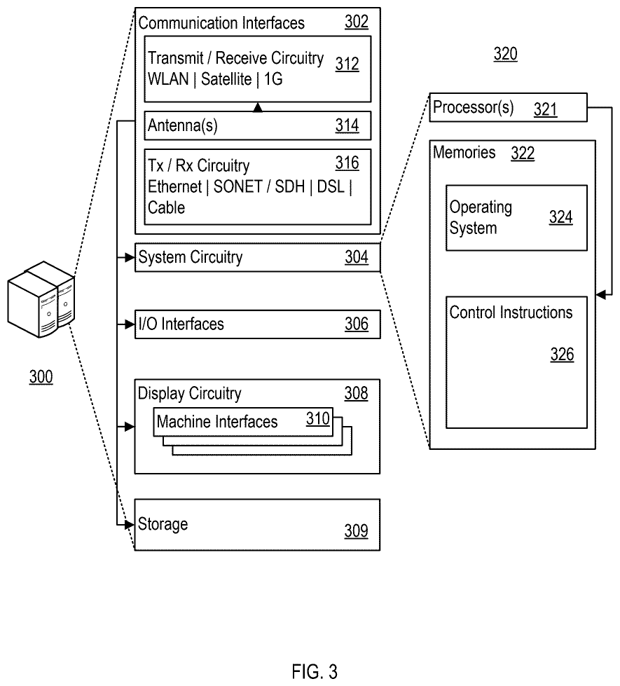

In some embodiments, FIG. 1 shows an apparatus (or an imaging system) 100 for acquiring a super-resolution image of a subject. The apparatus may include a portion or all of the following: a subject 110 may include a biological sample or a living subject; a modulator 120 may has a periodic pattern; a detector 130 may include a plurality of sensors, wherein each sensor may correspond to at least one pixel of a biomedical image; and/or a device 150 may include a processor 152 and a memory 154. The device 150 may include a portion or all of a suitable electronic device, for example, a computer as shown in FIG. 3.

The modulator 120 includes a periodic pattern configured to modify an original image field 115 before the original image field propagates onto the detector of the imaging system, the original image field originating from a subject and comprising information of the subject. The modulation of the modulator may be facilitated by physically moving (e.g., translational motion, rotational motion, or a combination of both translational and rotational motion), or electronically modulation (for example, absorption or transmission changes with a modulated voltage on the modulator). The detector 130 is configured to detect the modified original image field 125 to acquire a plurality of detected images. The memory 154 may store instructions and the processor 152 is in communication with the memory. The device 150 may be in communication with the detector by sending control signals and receiving image data from the detector. The device 150 may be in communication with the modulator by providing modulation/control instructions and/or receiving feedback signals from the modulator. When the processor executes the instructions, the processor is configured to cause the apparatus to reconstruct a super-resolution image based on the plurality of detected images, wherein an imaging resolution of the super-resolution image is better than an intrinsic resolution of the detector. In some implementations, the sensitivity pattern of the detector may be electronically modulated by applying a series of modulation voltages on the detector, for example, detectors for x-ray and/or gamma rays.

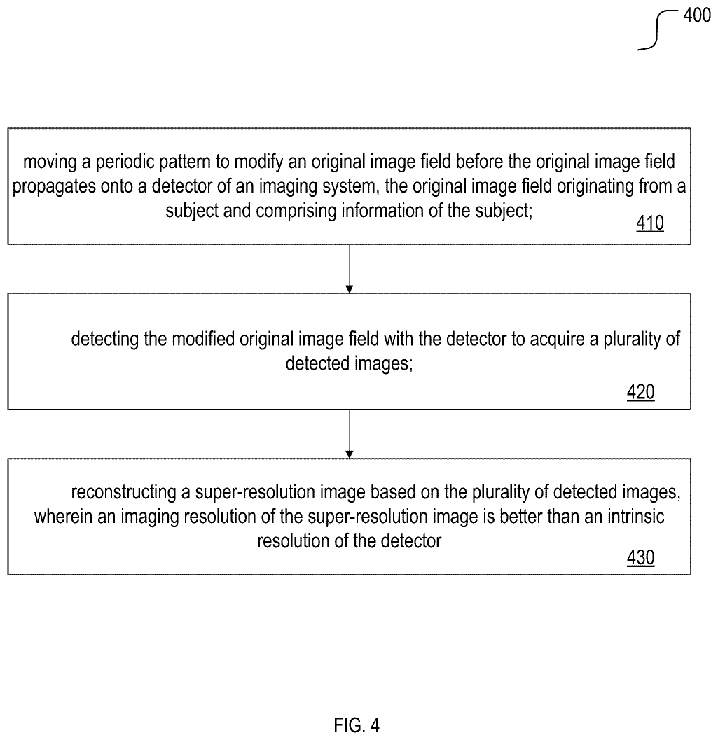

Referring to FIG. 4, various embodiments in the present disclosure may include a method 400 for acquiring a super-resolution image of a subject. The method 400 may include a portion or all of the following steps: step 410, moving a periodic pattern to modify an original image field before the original image field propagates onto a detector of an imaging system, the original image field originating from a subject and comprising information of the subject; step 420, detecting the modified original image field with the detector to acquire a plurality of detected images; and/or step 430, reconstructing a super-resolution image based on the plurality of detected images, wherein an imaging resolution of the super-resolution image is better than an intrinsic resolution of the detector. The method 400 may be performed by any suitable electronic device, for example, a computer as shown in FIG. 3.

In some implementations, the reconstructed super-resolution image has a higher spatial frequency than a spatial frequency of the imaging system.

In some implementations, the modulator may include a rotatable mask comprising the periodic pattern. The rotatable mask is configured to constantly rotate; and the detector is configured to constantly detect the modified original image field to acquire the plurality of detected images, wherein each of the plurality of detected images is indexed with information corresponding to a position of the periodic pattern when the detected image is acquired.

In some implementations, the modulator may include a rotatable mask comprising the periodic pattern; the rotatable mask is configured to rotate in a step-by-step manner; and the detector is configured to detect the modified original image field to acquire the plurality of detected images in a step-and-shoot manner corresponding to the rotatable mark, wherein each of the plurality of detected images is indexed with information corresponding to a position of the periodic pattern when the detected image is acquired.

In some implementations, the detector comprises at least one of the following: a charge coupled device (CCD) for detecting optical field, a positron emission tomography (PET) detector for detecting radiation field, or a single photon emission computed tomography (SPECT) detector for detecting radiation field.

In some implementations, the periodic pattern comprises one of the following: a structured absorptive pattern, or a structured sensitivity pattern. In some implementations, the periodic pattern comprises higher order harmonics. In some implementations, the periodic pattern has a spatial frequency higher than a spatial frequency of the imaging system. In some implementations, the periodic pattern comprises one or more periodic absorptive patterns. In some implementations, the periodic pattern comprises two orthogonal periodic absorptive patterns. In some implementations, the periodic pattern comprises a periodic multi-level absorption or sensitivity pattern.

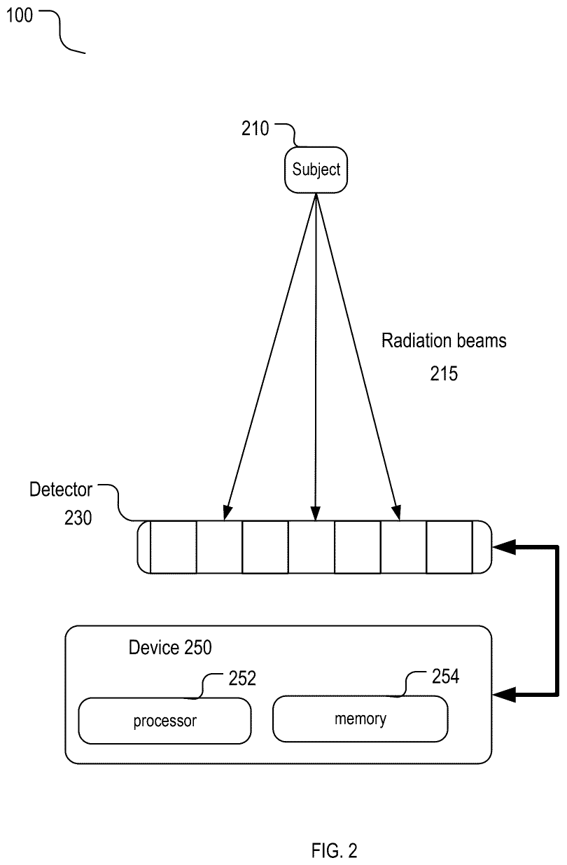

In some embodiments, FIG. 2 shows another apparatus (or another imaging system) 200 for acquiring a super-resolution image of a subject. The apparatus may include a portion or all of the following: a subject 210 may include a biological sample or a living subject; a detector 230 is capable of being modulated and may include a plurality of sensors, wherein each sensor may correspond to at least one pixel of a biomedical image; and/or a device 250 may include a processor 252 and a memory 254. The device 250 may include a portion or all of a suitable electronic device, for example, a computer as shown in FIG. 3.

The detector 230 may be capable of being modulated, and a modulation of the detector is configured to cause modulation of radiation beams 215, the radiation beams originating from a subject and comprising information of the subject, and/or the detector is configured to receive the radiation beam to acquire a plurality of detected images. In some implementations, the sensitivity pattern of the detector may be electronically modulated by applying a series of modulation voltages on the detector, for example, detectors for x-ray and/or gamma rays.

The memory 254 stores instructions and the processor 252 is in communication with the memory, wherein, when the processor executes the instructions, the processor is configured to cause the apparatus to reconstruct a super-resolution image based on the plurality of detected images, wherein an imaging resolution of the super-resolution image is better than an intrinsic resolution of the detector. The device 250 may be in communication with the detector by sending control signals and receiving image data from the detector, and/or providing modulation/control instructions and/or receiving feedback signals from the detector regarding modulation.

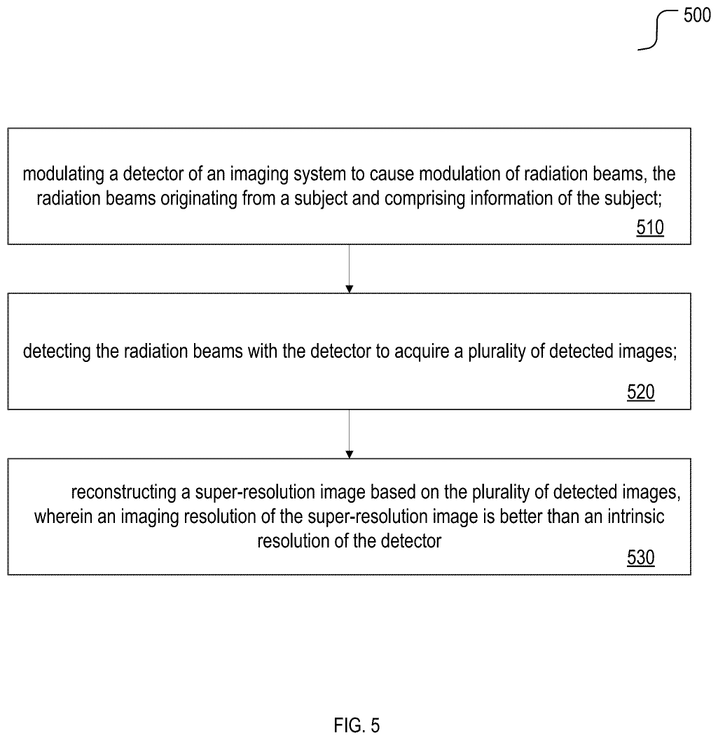

Referring to FIG. 5, various embodiments in the present disclosure may include another method 500 for acquiring a super-resolution image of a subject. The method 500 may include a portion or all of the following steps: step 510, modulating a detector of an imaging system to cause modulation of radiation beams, the radiation beams originating from a subject and comprising information of the subject; step 520, detecting the radiation beams with the detector to acquire a plurality of detected images; and/or step 530, reconstructing a super-resolution image based on the plurality of detected images, wherein an imaging resolution of the super-resolution image is better than an intrinsic resolution of the detector. The method 500 may be performed by any suitable electronic device, for example, a computer as shown in FIG. 3.

In some implementations, the reconstructed super-resolution image has a higher spatial frequency than a spatial frequency of the imaging system. In some implementations, the modulation of the detector is at a sensor level, wherein the detector comprises a plurality of position sensitive sensors, whose output is amplified with a different gain depending on a corresponding detection position with a sensitivity profile being modified electronically. In some implementations, the plurality of position sensitive sensors comprises at least one of the following: a position sensitive photo-multiplier tube (PMT), a position sensitive avalanche photo-diode (APD), a position sensitive Silicon photo-multiplier (SiPM).

Referring to FIG. 3, a computer system (electronic device) 300 may include communication interfaces 302, system circuitry 304, input/output (I/O) interfaces 306, storage 309, and display circuitry 308 that generates machine interfaces 310 locally or for remote display, e.g., in a web browser running on a local or remote machine. The machine interfaces 310 and the I/O interfaces 306 may include GUIs, touch sensitive displays, voice or facial recognition inputs, buttons, switches, speakers and other user interface elements.

The machine interfaces 310 and the I/O interfaces 306 may further include communication interfaces with modulators, sensors, and/or detectors. The communication between the computer system 300 and the sensors and detector may include wired communication or wireless communication. The communication may include but not limited to, a serial communication, a parallel communication; an Ethernet communication, a USB communication, and a general purpose interface bus (GPIB) communication. Additional examples of the I/O interfaces 306 include microphones, video and still image cameras, headset and microphone input/output jacks, Universal Serial Bus (USB) connectors, memory card slots, and other types of inputs. The I/O interfaces 306 may further include magnetic or optical media interfaces (e.g., a CDROM or DVD drive), serial and parallel bus interfaces, and keyboard and mouse interfaces. The quantum-classical interface may include a interface communicating with a quantum computer.

The communication interfaces 302 may include wireless transmitters and receivers (“transceivers”) 312 and any antennas 314 used by the transmitting and receiving circuitry of the transceivers 312. The transceivers 312 and antennas 314 may support Wi-Fi network communications, for instance, under any version of IEEE 802.11, e.g., 802.11n or 802.11ac. The communication interfaces 302 may also include wireline transceivers 316. The wireline transceivers 316 may provide physical layer interfaces for any of a wide range of communication protocols, such as any type of Ethernet, data over cable service interface specification (DOCSIS), digital subscriber line (DSL), Synchronous Optical Network (SONET), or other protocol. In another implementation, the communication interfaces 302 may further include communication interfaces with the modulators, sensors, and/or detectors.

The storage 309 may be used to store various initial, intermediate, or final data. In one implementation, the storage 309 of the computer system 300 may be integral with a database server. The storage 309 may be centralized or distributed, and may be local or remote to the computer system 300. For example, the storage 309 may be hosted remotely by a cloud computing service provider.

The system circuitry 304 may include hardware, software, firmware, or other circuitry in any combination. The system circuitry 304 may be implemented, for example, with one or more systems on a chip (SoC), application specific integrated circuits (ASIC), microprocessors, discrete analog and digital circuits, and other circuitry. For example, the system circuitry 304 may include one or more instruction processors 321 and memories 322. The memories 322 stores, for example, control instructions 326 and an operating system 324. In one implementation, the instruction processors 321 execute the control instructions 326 and the operating system 324 to carry out any desired functionality related to the controller.

The electronic device in the present disclosure and/or the methods described in the present disclosure may be implemented by a portion or all of the computer system 300 as described above. In some implementations, the modulations achieved by moving pattern may also be achieved by detectors that serve both to cause modulation of the radiation beams and at the same time captures event so that there is no loss in sensitivity. In some implementations, movement may be one way of achieving the needed modulation of the radiation beam, and the modulation may also be achieved at the sensor level, for example position sensitive PMT or APD or SiPM in which the output will be amplified with a different gain depending on the detection position and the corresponding sensitivity profile is moved for acquiring multiple measurements, much like the absorption pattern is moved. But this is done electronically; there is no actual movement.

Reconstruction of Super-Resolution Image with Modulations: Introduction

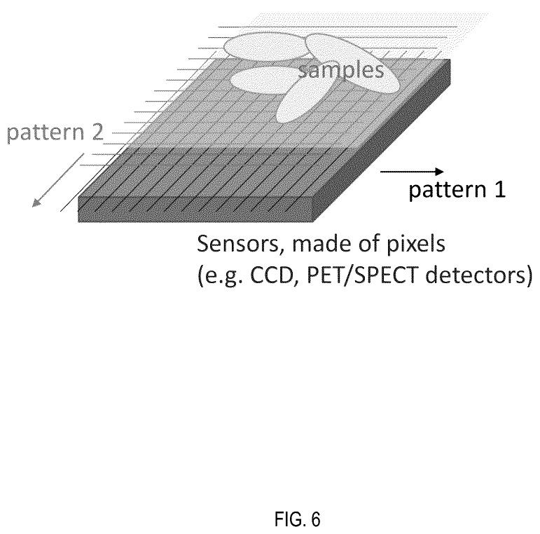

In some embodiments, as shown in FIG. 6, an imaging system includes a pixelated detector (or sensor) such as the CCD or CMOS CCD for light detection, and radiation detectors for PET or SPECT. There may be two orthogonal, periodic absorptive patterns of a certain fundamental period T. A set of images are taken with the absorptive patterns at various positions, by using step and shoot movements. It is also possible to use constant speed motions if the sensor is capable of (1) a high frame rate, or (2) photon counting in the case of radiation detector and produces list mode data (individual events stored in chronological order of detection). The images are solved together based on a mathematical model describing the imaging process to yield an image at a resolution beyond the sensor's intrinsic resolution.

In some implementations, a lens-less system may be includes. In some implementations, when needed or desired, optical components may be introduced between samples and the absorptive patterns, or between the absorptive patters and the sensor. In some implementations, the described techniques may be applied to implementing the moving structured absorption patterns before a camera to boost its resolution and/or enlarging its field of view. In some implementations, the descried mathematical model may be currently solved by a Fourier method, and optimization based iterative methods may be used for dealing with data noise and other nonideal issues. In some implementations, a critical function of the absorptive patterns is to multiply the image field by a periodic pattern before it enters the camera, which may be achieved by other means. For example, some novel ways to electronically modulate the sensitivity of the sensor.

Below, the present disclosure describes an example of using the described technology to restore/reconstruct resolutions on simulated data with a 2-step reconstruction process. The process may require separable camera response function, which is not unrealistic in real world situations. In some implementations, this 2-step process may be more computationally practical, even though it may not be the most optimal process.

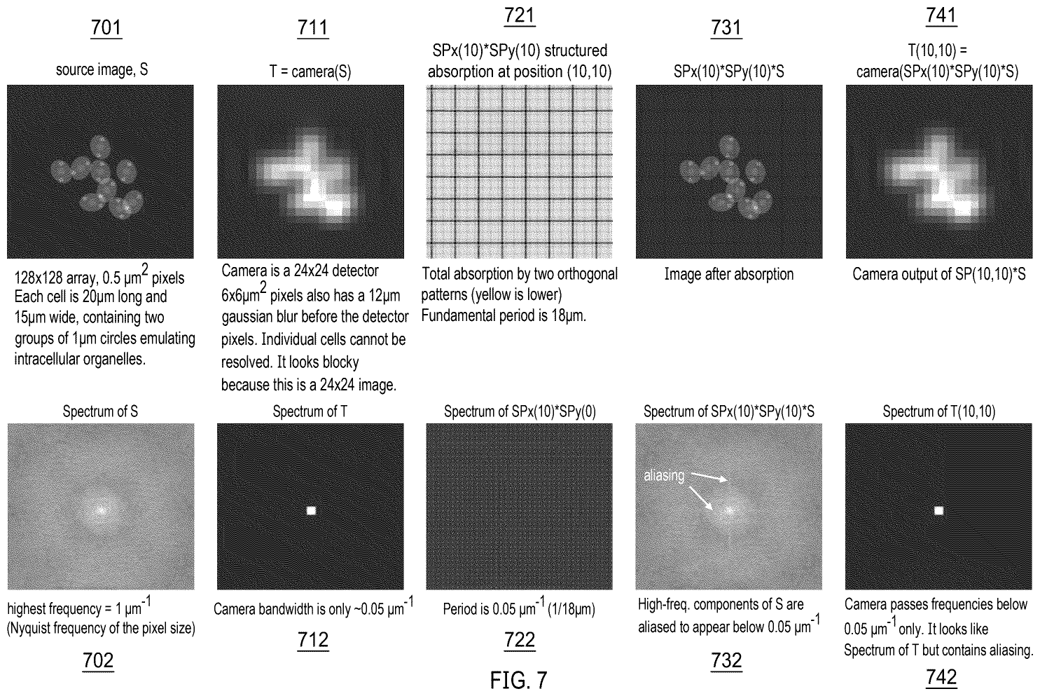

FIG. 7 shows charts of computer simulated data for various embodiments in the present disclosure. A source image(S) is displayed in 701 with its corresponding spectrum in 702. The source image has 128×128 array with 0.5 micron*micron per pixel. Each cell is about 20 micron long and 15 micron wide, containing two groups of 1 micron circles emulating intracellular organelles. The spectrum of the source image has a highest frequency of 1 micron⁻¹, which corresponds to Nyquist frequency of the pixel size.

Referring to camera output/response (T, 711) and its corresponding spectrum of T (712), the camera is a 24×24 detector with 6×6 micron*micron per pixel, and also with a 12 micron Gaussian blue before the detector pixels. The individual cells looks blocky and may not be resolved because this is a 24×24 image. The camera output/response may be denoted as T=camera(S). For the spectrum of T, the camera bandwidth is only about 0.05 micron⁻¹.

Referring to 721, a structured absorption is positioned at position (10,10) with SPx(10)*SPy(10), the total absorption by two orthogonal patterns (lighter color or lighter shade being lower in absorption), and fundamental period is 18 micron. The spectrum of SPx(10)*SPy(10) in 722 is shown with period being about 0.05 micron⁻¹ (which is about 1/18 micron⁻¹).

Referring to 731, an image after absorption is shown as SPx(10)*SPy(10)*S. Referring to 732, a spectrum of SPx(10)*SPy(10)*S is shown with aliasing as indicated by arrows, wherein the high frequency components of S are aliased to appear below 0.05 micron⁻¹.

Referring to 741, the camera output/response of SPx(10)*SPy(10)*S is shown. The camera output/response may be denoted as T(10,10)=camera (SPx(10)*SPy(10)*S). Referring to 742, the spectrum of T(10,10) is shown, wherein the camera passes frequencies below 0.05 micron⁻¹ only, it looks like spectrum of T only, but contains additional aliasing information.

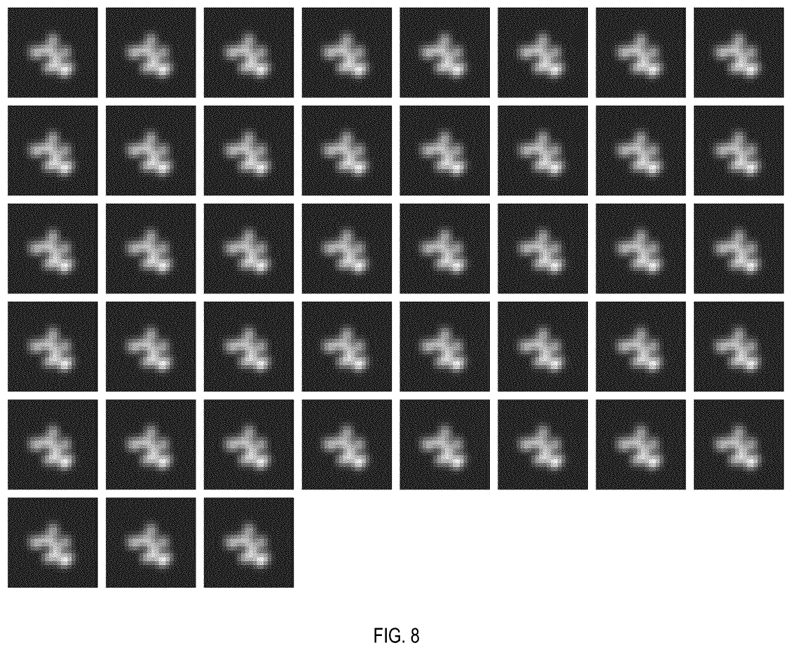

FIG. 8 shows charts of computer simulated data at 43 different positions for various embodiments in the present disclosure. 43 x and y positions are used for the structured patterns SPx and SPy, respectively. The 43 camera output/response images when moving the SPx pattern over 43 positions while fixing the SPy pattern at position 0 are shown, wherein T(x,0)=SPx(x)*SPy(0)*S. While each of the 43 camera output images may look quite similar to each other, they in fact contain subtle differences that allow resolution recovery/reconstruction.

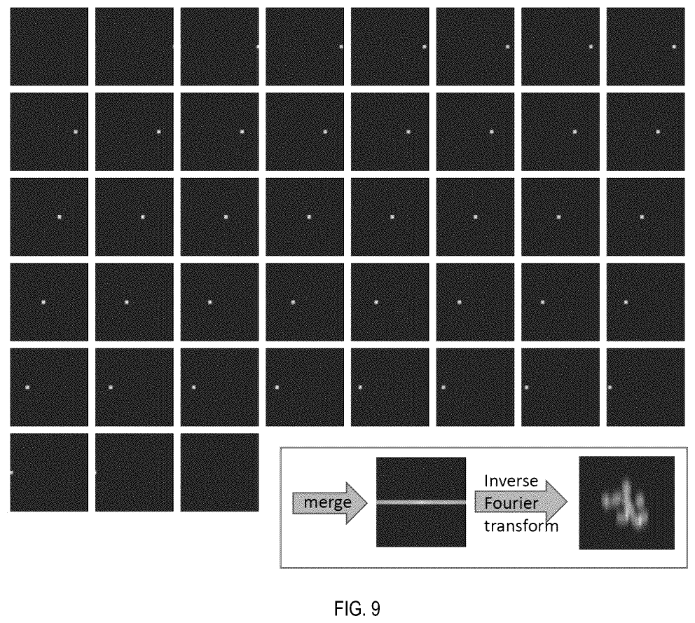

FIG. 9 shows charts of estimated spectra at 43 different positions for various embodiments in the present disclosure. the estimated spectra of camera (SPy(0)*S), wherein the camera output of SPy(o)*S in 43 different x-frequency bands from SPx(x)*SPy(0)*S images shown in FIG. 8. The spectra are merged to recover the spectrum of camera (SPy(0)*S) over the full x-frequency range, wherein y-frequency range is still limited by the camera bandwidth. The images now looks sharper, and cell may start to be identifiable with a hint of the existence of intracellular organelles.

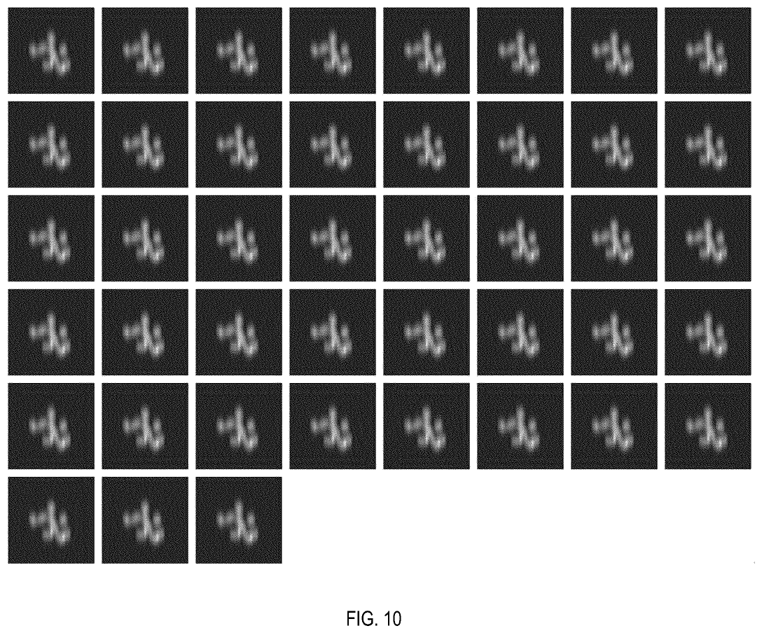

FIG. 10 shows charts of images for various positions for various embodiments in the present disclosure. The 43 estimated camera (SPy(y)*S0 obtained by repeating the above procedure to the camera outputs obtained at 43 y positions of the SPy patterns.

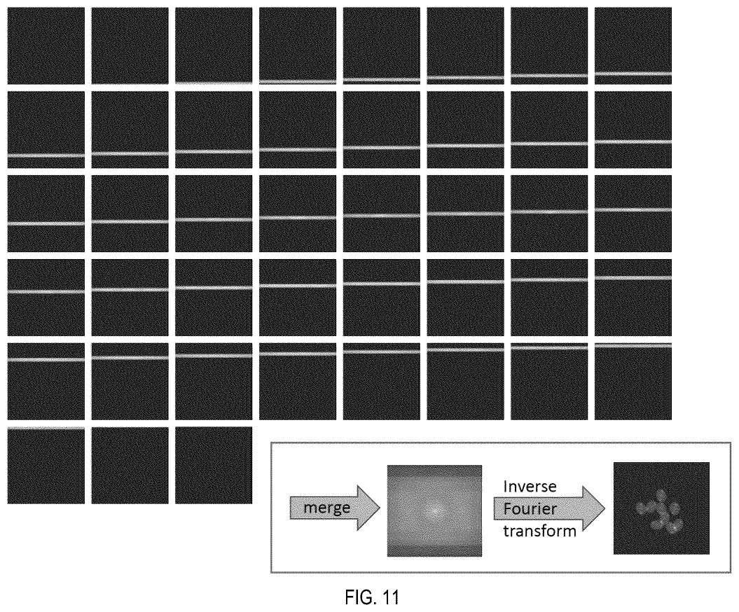

FIG. 11 shows charts of spectra for various positions for various embodiments in the present disclosure. The spectra of S in 43 y frequency bands obtained from camera (SPy(y)*S) are merged to obtain the spectrum of S over the entire frequency range. The resulting image at lower right corner shows full restored resolution.

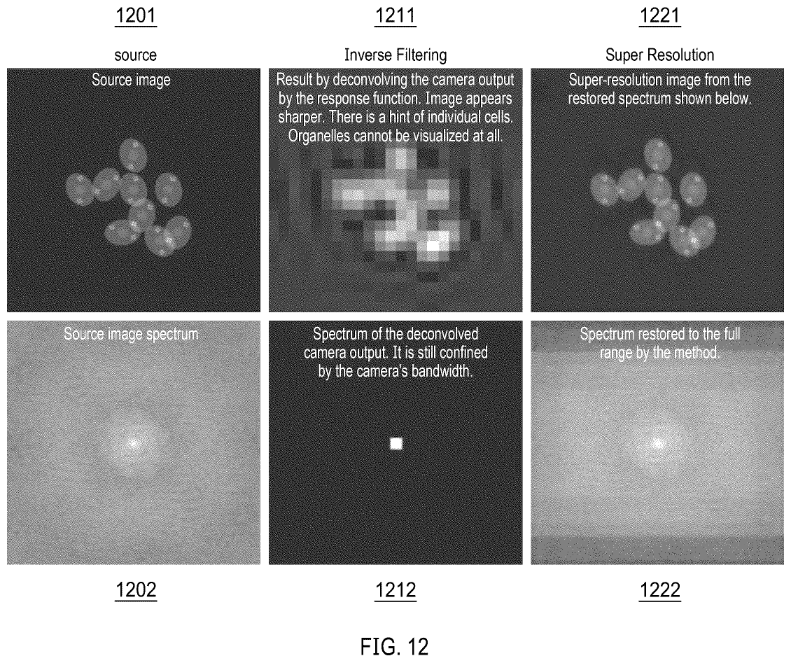

FIG. 12 shows examples of reconstructing process for various embodiments in the present disclosure. A simulated source image is shown in 1201, and its spectrum is shown in 1202. For 1211, an inverse filtering process is shown, wherein result by deconvolving the camera output by the response function. The image in 1211 appears sharper than a pure camera response with 24×24 pixels. The image in 1211 shows a hint of individual cells, although organelles still can not be visualized at all. For 1212, a spectrum of the deconvolved camera output is shown, which is still confined by the camera's bandwidth. For 1221, a super resolution image is shown, which is from the restored spectrum in 1222. The spectrum in 1222 is restored to the full range by the technique described above.

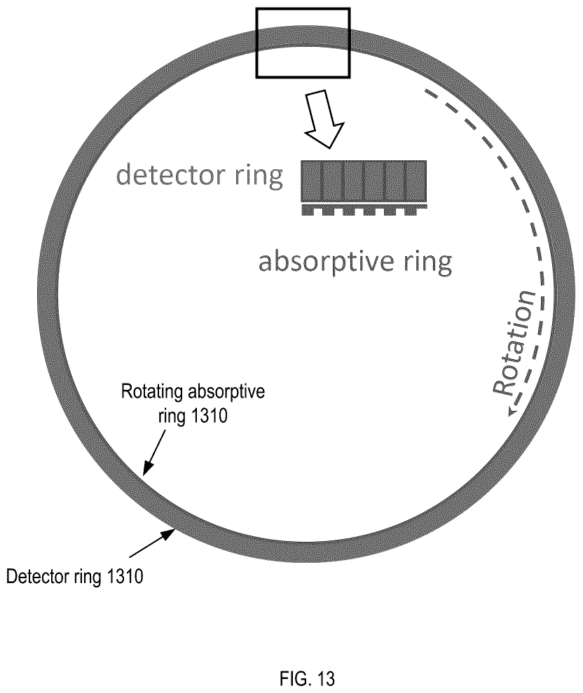

The technique described above may be applicable to many biomedical imaging modalities. For a non-limiting example, such an application for super resolution (SR) in position emission tomography (PET) is described below. Referring to FIG. 13, a clinical PET detector ring 1310 may be capable of 2 mm and some preclinical detector ring may be capable of <1 mm. In some implementations, the resolution of clinical PET system remains 4 to 6 mm even though it has been recognized as insufficient for some applications, such as brain imaging. High resolution detectors are expensive and scaling these technologies to develop clinical systems is a nontrivial and costly process. The described technique in the present disclosure provides an immediate and inexpensive way to boost the resolution of an existing clinical system without modifying the instrumentation to 2 mm or better. In some implementations, with the advent of time of flight (TOF) PET, as detectors capable of simultaneous high TOF and spatial resolutions are difficult to develop and expansive, this allows one to focus on optimizing the TOF resolution while relaxing the requirement on the spatial resolution. A rotating absorptive ring 1320 includes an absorption pattern, and when it's rotating, it modulates the imaging field before reaching to the detector ring. The rotating absorptive ring can be implemented as a replaceable add-on to the system. Different absorptive rings may be developed and used to meet the different needs of different imaging applications without requiring making changes to the core instrument.

### Super-Resolution Embodiments: Super-Resolution PET

In various embodiments, the principle of structured illumination microscopy (SIM) may be used for increasing the resolution of a clinical PET system, wherein the PET projection data may be modulated before detection to encode image information above the resolution limit of a PET system into lower-resolution signals so that the information is present in the acquired data. Subsequently, from multiple such modulated limited-resolution measurements, in accordance with the measurement model one can compute an image whose resolution is beyond the intrinsic resolution of the system. At least two methods may be used for creating modulations. One method include using a patterned absorption ring (PAR) whose diameter matches the inner diameter of the PET detector ring (DR). This PAR is rotated at a constant low speed and synchronized to acquisition so that the resulting list-mode data can be divided into measurements made at a number of discrete rotational positions. PAR is different from collimator: The former only partially absorbs radiations to create modulation but the latter fully stops radiations to define lines of sight. Nonetheless, imaging sensitivity is still reduced by the presence of PAR. Another method may include using a rotating inner detector ring (IDR) to both create modulation and detect events. In some implementations, the IDR may be low-resolution so that it is not expensive or difficult to fabricate.

PET is an in vivo imaging modality known for its ability to reveal molecular processes underlying diseases with high sensitivity and high specificity. In some implementations, compared to computed tomography and magnetic resonance imaging, the resolution of the state-of-the-art clinical PET systems is subpar, only 4 mm at best. Unsurprisingly, this resolution is determined by the need of the predominant clinical application of PET, which is whole-body cancer imaging. It is however insufficient for, for example, detecting early-stage lesions and imaging specific organs or regions such as the head-and-neck, pancreas and brain that have important small structures or delicate anatomy. In some implementations, specifically, <2 mm image resolution is necessary for the brain. Although submillimeter-resolution detectors are now available and used in preclinical systems, the task of improving the resolution of clinical PET systems is complicated by the competing need in sensitivity and market factors. Some implementations have been on enabling and improving time-of-flight (TOF) detection for increasing imaging sensitivity so that better quantification, lower dose, and shorter scan (better temporal resolution in dynamic studies) can be achieved. In some implementations, recent development were made for long-bore systems whose axial FOVs (AFOV) can cover the entire torso. Due to their large solid angles and TOF detection, these systems have an order of magnitude higher sensitivity than their shorter cousins, thereby allowing longer acquisition delays to increase tumor-to-normal tissue contrast and ultrafast acquisition to minimize motion-induced degradations. Its extended AFOV is useful for detecting distant metastases and vascular complications, and imaging multiorgan diseases. A fundamental issue in improving clinical PET resolution is that higher detector resolution does not automatically lead to higher system resolution. The uncertainty about the depth into the detector where a gamma ray interacts leads to depth-of-interaction (DOI) blurring that degrades the axial resolution and progressively degrades the transverse resolution with the radial distance. In some implementations, improving detector resolution generally increases DOI blurring also unless detector thickness is reduced. Thus, to maintain detection efficiency, high-resolution detectors must also be capable of measuring DOI with accuracy. Therefore, high-resolution, TOF/DOI-capable detectors is ultimately needed and their development has been an active research topic over the past several decades. Although impressive progresses have been made, in view of the already high system cost, cost effectiveness will be a strong factor affecting their adoption into clinical systems. Alternatively, dedicated systems having higher resolutions for specific organs/regions, particular for the brain, have been proposed. Unfortunately, these systems may not be widely available due to their limited market size. Therefore, there are some issues wherein the resolution of clinical PET at most sites is unlikely to improve beyond its current level, and continue to be inadequate for many important applications.

In some implementations, a resolution of a given PET system can be improved by better image reconstruction. A PET system may be regarded as a linear shift-invariant (LSI). At the center of the FOV, the detector response function is a triangle whose base equals the detector width d. In frequency space, the magnitude of this response rolls off to reach its first zero at 2/d, which is often taken as its cutoff frequency, or bandwidth, v_{B}. Image reconstruction methods that take into account the detector response can compensate for the roll-off to restore the resolution, up to v_{B}. However, this discussion has not considered sampling. Nowadays, nearly all clinical PET systems are stationary systems and are known to produce under-sampled data in the central FOV region. Specifically, there the Nyquist frequency of sampling is only 1/d or v_{B}/2; thus, the highest frequency that can be restored decreases to v_{B}/2. When aliasing errors are present, this frequency is further reduced because small aliasing errors near v_{B}/2 will be substantially amplified. Therefore, to recover image resolution up to v_{B}, sampling must be increased. In some implementations, detector motions (raising stability concerns as moving detectors and electronics are more prone to failure), patient-bed translations (raising concerns for patient comfort and safety), DOI detectors (not used in present clinical systems), and some other means may be used. In some implementations, Methods for increasing sampling and detector resolution response for regions of interest are also used, such as introducing high-resolution insert detectors into the FOV of a limited-resolution system and building a system using detectors having different resolutions. These approaches may be less expensive, but their implementations are still nontrivial. In some implementations, moreover, they boost resolution only locally. In some implementations, using inserts can considerably decrease the FOV.

In some implementations, the resolution of a PET system may be improved by using collimation to decrease the detector width if it is accompanied by a commensurate increase in sampling to avoid under-sampling. This approach can increase the image resolution to v_{B}=2/d′, where d′ is the detector width after collimation. In some implementations, a specific collimator design may be used for yielding d′=d/2. In addition to reducing the imaging sensitivity of the PET system by as much as 75 percent, the proposed collimator movements for increasing sampling were rather difficult to implement. In some implementations, despite substantial sensitivity loss and increased image noise, higher contrast recovery and lower mean square errors may be achieved for small structures.

In some implementations, while the above-reviewed RE methods improve the resolution of the images produced by a system by restoring the suppressed high-resolution signals up to the bandwidth of the system or the Nyquist frequency of sampling, SIM not only restores the suppressed signals but also effectively expands the bandwidth of the system. The classical (or linear) SIM may double the bandwidth and the nonlinear SIM in theory may expand the bandwidth indefinitely. The basic theory of SIM is described in the present disclosure. In some implementations, the RE methods that create over-sampled data to allow restoration of image resolution up to v_{B} may be often called SR as well.

In some implementations, the effects of the structured illumination in the theory of SIM may be achieved by absorption of radiation beams before their detection, and the resolution of a PET system may be boosted beyond its instrumentation limit. As already discussed above, the cutoff frequency of a stationary PET system due to instrumentation is about v_{B}/2=1/d. The conventional SIM theory does not consider sampled data. In some implementations, SIM can also eliminate aliasing errors associated with under-sampling. The equivalent of linear SIM implemented for PET therefore can recover image resolution up to 2v_{B}=4/d, corresponding to a 4-fold increase. For the present-day clinical systems, this means increasing the resolution from 4 mm to 1 mm. A 1 mm image resolution is favorable for the brain and is higher than most dedicated systems proposed to date. An even higher resolution may be achieved with nonlinear SIM.

Various embodiments in the present disclosure may achieve surpassing the resolution limit of a PET system due to instrumentation, and can be applied to other radiative imaging modalities. In some implementations, PARs can be implemented as relatively inexpensive and easy-to-use add-one devices for a pre-existing clinical PET system without requiring changes to its instrumentation or the patient bed. It also minimally affects the imaging FOV. In some implementations, the concept of modulating IDRs can be used to create new design concepts for developing high-end PET systems. For example, in situation when resources are limited one may invest them to optimize the TOF resolution and/or maximize the axial FOV while allowing some compromises in the detector resolution.

Super-Resolution Embodiments: Imaging with Undersampled Data

The present disclosure describes various embodiments wherein measurement is undersampled and a classical theory for treating data of this kind is extended. The classical theory of structured illumination microscopy (SIM) for super-resolution imaging is formulated for continuous measurement but modern imaging devices produce sampled measurement. Under suitable conditions, the extension can be made, allowing both elimination of aliasing errors due to undersampling and recovery of signal beyond the bandwidth of the imaging device. A link to Papoulis' generalized sampling theorem is provided, and numerical experiments are shown to validate the extended theory.

### INTRODUCTION

Structured illumination microscopy (SIM) is one of the super-resolution (SR) methods developed to surpass the intrinsic resolution limit in microscopy due to diffraction of lights. It uses modulation to shift down the components of a signal above the bandwidth (BW) of the imaging device to appear within and thereby become detectable, albeit in an aliased form. The measured signal is then computationally de-aliased to recover the original signal to an extent beyond the device's BW. The basic theory considers continuous measurement but in practice sensors such as the charge-coupled devices (CCD) that consist of discrete detection elements, called pixels, are used. In microscopy, when designed properly the pixel size is made sufficiently small with respect to the point spread function (PSF) of the optics on the focus plane so that sampling aliasing is absent. There are important applications in which the effects of sampling cannot be ignored. In the present disclosure, the basic SIM theory for undersampled discrete measurement is extended, showing that under suitable conditions the theory remains applicable and aliasing errors due to undersampling can be eliminated. Numerical studies are conduct to validate the theory.

### Notations and Conventions

Below, a spatial function is denoted by a lower-case letter (e.g. ƒ(x)) and its Fourier transform (FT) by the corresponding upper-case letter (e.g., F(ν)). A function and its FT are treated as the same as used interchangeably. A function ƒ(x) is said to be σ-bandlimited (essentially σ-bandlimited), or to have a BW (an essential BW), if F(ν) is zero (negligibly small) for |ν|>σ and nonzero for |ν|<σ. Given a number z, its complex conjugate is denoted by z*. The notations {aₖ [symbol] and {aₖ}_{k=-K}^{K} are shorthands for {aₖ:k∈ [symbol] } and {aₖ:−K≤k≤K}, respectively. A matrix is written by using a scripted uppercase letter such as [symbol] , or as [aᵢⱼ] where aᵢⱼ is the element at row i and column j. Given samples {ƒ(nd) [symbol] of a function ƒ(x) for some sampling distance d>0, one can define an associated sampling function by ƒ^{s}(x)= [symbol] ƒ(nd)δ(x−nd). A linear operation on {ƒ(nd) [symbol] has an equivalent linear operation on ƒ^{s}(x) and vice versa.

### Theory

### Classical SIM Theory

For completeness and to establish terms, conditions and background for subsequent treatments, a concise overview of the classical SIM theory is described first. Let ƒ(x) be the input signal and h(x) the point spread function (PSF) of a linear shift-invariant (LSI) imaging device that has a BW σ; i.e., h(x) is σ-bandlimited. Let m(x) be a real periodic function whose frequency is vo. Then, it has a Fourier series expansion (FSE) given by

$$m ( x ) = {\sum }_{k = - K}^{K} {m}_{k} {e}^{j 2 π k {ν}_{0} x} with {m}_{- k} = {m}_{k}^{*} \qquad ( 1 )$$

for some K>0. In SIM, ƒ(x) is modulated by m(x− [symbol] ) before imaging to yield the measurement

$${g}_{ℓ} ( x ) = h ( x ) ⋆ \{ m ( x - {x}_{l} ) f ( x ) \} , \qquad ( 2 )$$

where ★ denotes convolution. Taking FT of both sides of Eq. (2) and using Eq. (1),

$${G}_{ℓ} ( ν ) = {\sum }_{k = - K}^{K} {c}_{ℓ k} {F}_{k} ( ν ) , \qquad ( 3 )$$

where cₗₖ=mₖ [symbol] and

$${F}_{k} ( ν ) = H ( ν ) F ( ν - k {ν}_{0} ) . \qquad ( 4 )$$

Evidently, Fₖ(ν)= [symbol] (ν)=0 for |ν|>σ due to H(ν). Assume that 2K+1 measurements are obtained by using 2K+1 translations { [symbol] creating a system of equations that relates {Fₖ(ν)}_{k=-K}^{K} to {Gₗ(ν) [symbol] with a coefficient matrix [symbol] = [symbol] Provided that | [symbol] |≠0, one can solve {Fₖ(ν)}_{k=-K}^{K} from { [symbol] (ν) [symbol] for every |ν|≤σ. Subsequently, one can compute F(ν−kν₀)=H⁻¹(ν)Fₖ(ν) for |ν|≤σ, or F(ν) for ν∈Ωₖ=[kν₀−σ, kν₀+σ]. If

$${ν}_{0} \leq 2 σ , \qquad ( 5 )$$

then kν₀+σ≥(k+1)ν₀−σ and adjacent Ωₖ's overlap. Thus, F(ν) has been determined up to the frequency

$${σ}_{x} ≡ K {ν}_{0} + σ . \qquad ( 6 )$$

The above analysis remains valid whenever there exist 2K+1 measurements that can be described by Eq. (3) and the resulting system of equations is invertible. In particular, one may use 2K+1 different mₗ(x)=Σₖ [symbol] e^{j2πkν}^{₀}^{x} and so [symbol] = [symbol] . One advantage of using regular translations of m(x) with [symbol] = [symbol] δx, δx=[(2K+1)ν₀]⁻¹ is that a Wiener-filter type closed-form solution exists. It is given by

$$F ( ν ) = {\sum }_{k = - K}^{K} \frac{{m}_{k}^{*} {H}^{*} ( ν + k {ν}_{0} ) {Y}_{k} ( ν + k {ν}_{0} )}{{\sum }_{k = - K}^{K} {\vert {m}_{k} H ( ν + k {ν}_{0} ) \vert}^{2} + α} , \qquad ( 7 )$$

where Yₖ(ν)= [symbol] (ν) [symbol] and α>0 is a parameter for regularization of noise.

Hence, under suitable conditions the BW of an imaging device can be expanded by solving a system of equations that relate multiple measurements made by the device when applying different modulations (conventionally achieved by translating a modulation pattern) to the image. Generally, for achieving a greater expansion, more measurements are needed. Since there is no theoretical limit on K, in principle the expansion can be infinite. In reality, only a finite number of measurements is possible. The achievable expansion also depends on the stability in inverting [symbol] , which is affected by the characteristics of the modulations, the inversion algorithm, and data noise.

In the above analysis, the highest harmonic order K of the modulation determines the number of terms on the right-hand side (RHS) of Eq. (3) and there is no assumption about the BW of ƒ(x). If ƒ(x) is (essentially) σₓ*-bandlimited where

$${σ}_{x}^{*} ≡ ( {K}^{*} + 1 ) {ν}_{0} - σ , {K}^{*} < K , \qquad ( 8 )$$

so that Fₖ(ν)=H(ν)F(ν−kν₀)≃0 for all |k|≥K*+1, then Eq. (3) has 2K*+1 terms on the RHS. In this case, the highest frequency covered by the solution is σ′=K*ν₀+σ. Under Eq. (5), σ′≥σₓ* and so ƒ(x) can be fully recovered.

Note that in the above discussion if the number of measurements exceeds 2K+1 or 2K*+1, the systems of equations are over-determined and still can be solved. To summarize, using a modulation whose highest harmonic order is K and whose frequency ν₀ is no greater than 2σ where σ is the BW of the imaging device, the SIM method allows determination of a signal for |ν|<σₓ=Kν₀+σ from 2K+1 or more measurements, and of a (essentially) σₓ*-bandlimited signal where σₓ*=(K*+1)ν₀−σ with K*<K from 2K*+1 or more measurements.

### Extension for Undersampled Measurement

The above considers continuous measurement [symbol] (x). In the modern era of digital imaging, sensors made of discrete detection elements called pixels are used to produce samples { [symbol] (nd) [symbol] with some d>0. Using the Shannon's sampling theory and Eq. (3), the FT of the sampling function [symbol] (x) associated with { [symbol] (nd) [symbol] is given by

$${G}_{ℓ}^{s} ( ν ) = {\sum }_{n = - ∞}^{∞} {G}_{ℓ} ( ν - n {ν}_{s} ) = {\sum }_{k = - K}^{K} {c}_{ℓ k} {F}_{k}^{s} ( ν ) , \qquad ( 9 )$$

where νₛ=1/d is the sampling frequency, and

$${F}_{k}^{s} ( ν ) = {\sum }_{n = - ∞}^{∞} {F}_{k} ( ν - n {ν}_{s} ) \qquad ( 10 )$$

is the FT of the sampling function ƒₖ^{s}(x) associated with {ƒₖ(nd) [symbol] Trivially, Fₖ(ν) and hence [symbol] (ν) are periodic with the period νₛ. Hence, with sampling the BW of the device shall be taken as

$${σ}_{s} = {ν}_{s} / 2 \qquad ( 11 )$$

in the sense that all information is available within σₛ. However, if the condition νₛ≥2σ is not satisfied, Fₖ(ν−nνₛ) in Eq. (10) overlap to create aliasing for νₛ−σ<|ν|≤σₛ, decreasing the meaningful BW to νₛ−σ≤νₛ/2.

In microscopy, lights are focused onto an imaging plane and the inherent PSF is due to diffraction of light that prevents perfect focus. Using magnification, at the imaging plane this PSF can be made sufficiently large with respect to the sensor's pixel size to satisfy the sampling condition. However, it is difficult or impossible to focus x- or γ-ray photons. Recently, there is also growing interest in lensless optical imaging. For these situations, the sampling condition to avoid aliasing is often violated. To illustrate, consider a linear array of pixels with no gap and assume that a pixel detects all photons entering its aperture. If i(x) is the photon fluence at this linear sensor, the measurement made by pixel n is

$${{y}_{n} = {\int }_{n d - d / 2}^{n d + d / 2} i ( x ) dx = i ( x ) ⋆ ⊓ ( x / d ) \vert}_{x = n d} , \qquad ( 12 )$$

where d and nd are, respectively, the pixel size and pixel position, and [symbol] (x)=1 for |x<½ and [symbol] (x)=0 otherwise. Therefore, the response function of the sensor is h(x)= [symbol] (x/d) whose FT is H(ν)=d·sin(πνd)/(πνd). Although there does not exist a σ such that H(ν)=0 for |ν|>σ, the magnitude of H(ν) decays rapidly as |ν| increases and a common practice is to use the first zeros (or second zeros) of H(ν) as its effective BW. This yields σ=1/δx=νₛ (or σ=2νₛ), violating the sampling condition.

However, it may be shown below that, when ν₀=pνₛ, p∈ [symbol] (case 1) and νₛ=qν₀, q∈ [symbol] (case 2) the sampling aliasing manifests in a similar way to the modulation aliasing created by SIM so that it also can be computationally de-aliased with the SIM framework. Due to the periodicity of [symbol] (ν), it is sufficient to consider ν∈Iₛ=[−νₛ/2, νₛ/2] in solving Eq. (9). Since Fₖ(ν) is σ-bandlimited, terms in Eq. (10) that vanish in Iₛ can be eliminated. This yields

$${F}_{k}^{s} ( ν ) = {\sum }_{n = - N}^{N} {F}_{k} ( ν - n {ν}_{s} ) for ν ∈ {I}_{s} , \qquad ( 13 )$$

$$where$$

$$N = ⌊ σ / {ν}_{s} + 1 / 2 ⌋ . \qquad ( 14 )$$

Note that N≥1 because νₛ<2σ by hypothesis. Also, N is a small integer unless Υ>>νₛ (strongly undersampled).

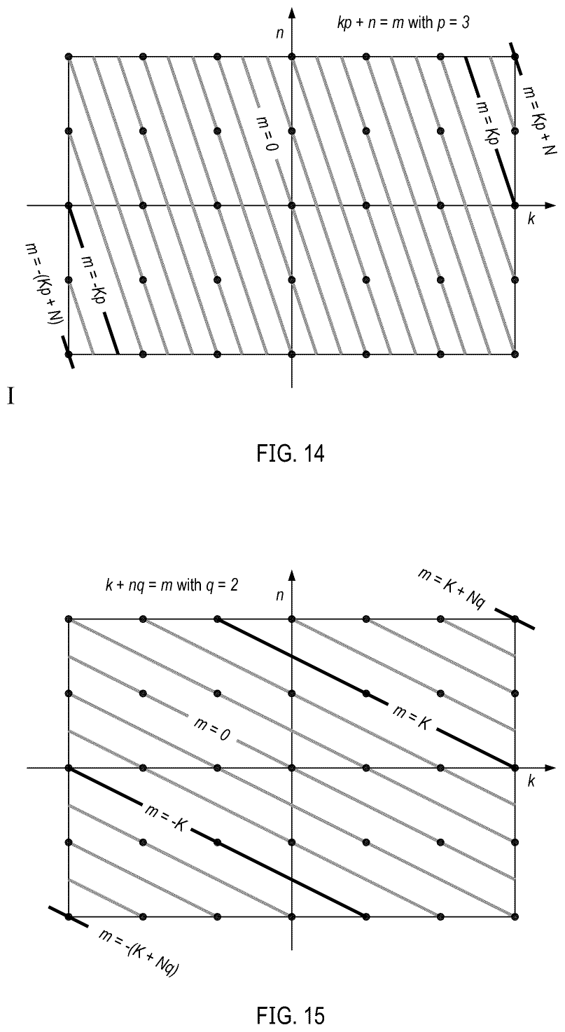

As shown in FIG. 14, Eqs. (9) and (13) in case 1 can be written as

$${G}_{ℓ}^{s} ( ν ) = {\sum }_{m = - {M}_{1}}^{{M}_{1}} {c}_{ℓ m} ( ν ) F ( ν - m {ν}_{s} ) for ν ∈ {I}_{s} \qquad ( 15 )$$

$$with$$

$${c}_{ℓ m} ( ν ) = {\sum }_{( n , k ) ∈ {ℒ}_{Γ} ( m )} {c}_{ℓ k} H ( ν - n {ν}_{s} ) , \qquad ( 16 )$$

where M₁=Kp+N and [symbol] _{Γ}(m) is the line segment given by the intersection of the line [symbol] (m)={(k, n):kp+n=m} with the region Γ={(k, n):|k|≤K, |n|≤N}. Now, as with Eqs. (3) & (4), if | [symbol] (ν)|≠0, ∀ν∈Iₛ, where [symbol] (ν)=[ [symbol] (ν)], one can solve {F(ν−mνₛ)}_{m=-M}_{₁}^{M}^{₁}, ∀ν∈Iₛ; i.e, F(ν) for ν∈Ω₁=∪_{m=-M}_{₁}^{M}^{₁}Ω_{1,m} where Ω_{1,m}=[(m−½)νₛ, (m+½)νₛ], yielding Ω₁={ν:|ν|≤(M₁+½)νₛ}. In some other sections, if F(ν−mνₛ)≈0, ∀ν∈Iₛ whenever |m|≥K*+1 with K*<M₁; i.e., if ƒ(x) is (essentially) bandlimited to (K*+1)νₛ−νₛ/2, then Eq. (15) involves only 2K*+1 terms and can be solved by using 2K*+1 or more measurements. This BW equals the highest frequency of the solution.

Likewise, as shown in FIG. 15, Eqs. (9) and (13) in case 2 can be written as

$${G}_{ℓ}^{s} ( ν ) = {\sum }_{m = - {M}_{2}}^{{M}_{2}} {c}_{ℓ m}^{′} ( ν ) F ( ν - m {ν}_{0} ) for ν ∈ {I}_{s} \qquad ( 17 )$$

$$with$$

$${c}_{ℓ m}^{′} ( ν ) = {\sum }_{( n , k ) ∈ {ℒ}_{Γ}^{′} ( m )} {c}_{ℓ k} H ( ν - n {ν}_{s} ) , \qquad ( 18 )$$

where M₂=K+Nq and [symbol] _{Γ}′(m)= [symbol] ′(m)∩Γ with [symbol] ′(m)={(k, n):k+nq=m}. Let [symbol] ′(ν)=[cₗₘ′(ν)]. If | [symbol] ′(ν)|≠0, ∀ν∈Iₛ, one can solve {F(ν−mν₀)}_{m=-M}_{₂}^{M}^{₂}, ∀ν∈Iₛ, or F(ν) for ν∈Ω₂=∪_{m=-M}_{₂}^{M}^{₂}Ω_{2,m} where Ω_{2,m}=[mν₀−νₛ/2, mν₀+νₛ/2], yielding Ω₂={ν:|ν|<M₂ν₀+νₛ/2}. Again, a signal (essentially) bandlimited to (K*+1)ν₀−νₛ/2 with K*<M₂ can be determined from 2K*+1 or more measurements.

Additional conditions arising from the requirement | [symbol] (ν)|≠0 and | [symbol] ′(ν)|≠0 for all ν∈Iₛ are discussed in the Appendix. In summary, the core idea of SIM is valid for sampled data obtained at a frequency νₛ<2σ where σ is the BW of the imaging device if the frequency of modulation ν₀ is given by (case 1) ν₀=pνₛ with

$$1 \leq p \leq ⌊ 2 σ / {ν}_{s} ⌋ , p ∈ {ℤ}^{+} , \qquad ( 19 )$$

or (case 2) νₛ=qν₀ with q∈ [symbol] +. In both cases, Eq. (5) is satisfied. Let K be the highest harmonic order of modulation, N be given by Eq. (14), M₁=Kp+N, M₂=K+Nq, M₁*=M₁−1 and M₂*=min {M₂, K+┌σ/νₛ−q/2┐q−1}. For case i=1 or 2, from 2K*+1 or more measurements where K*≤Mᵢ*, the method can determine signals that are (essentially) bandlimited to

$${σ}_{s , x}^{*} = ( {K}^{*} + 1 ) \times min \{ {ν}_{0} , {ν}_{s} \} - {ν}_{s} / 2. \qquad ( 20 )$$

For case 2, if M₂*=M₂, a signal can be determined for |ν|≤σ_{s,x}, where σ_{s,x}=M₂ν₀+νₛ/2, from 2M₂+1 or more measurements regardless if it is σ_{s,x}-bandlimited.
A Perspective from the Generalized Sampling Theorem

In some implementations, a generalized sampling theory (GST) may be used to determine a bandlimited function from samples of the outputs of a number of LSI systems that receive the function as a common input. Below, the theorem is rephrased in a more convenient form for treating the problem under consideration.

Let rₘ(x) be the response functions of 2M+1 LSI systems for −M≤m≤M. Suppose that the outputs of these systems with input ƒ(x) can be expressed as

$${y}_{m} ( x ) = {\int }_{- σ}^{σ} {R}_{m} ( ν ) F ( ν ) {e}^{j 2 π ν x} d ν , - M \leq m \leq M , \qquad ( 21 )$$

for some σ>0. Consider the following system of equations:

$${\sum }_{m = - M}^{M} {R}_{m} ( ν + {ℓν}_{s} ) {W}_{m} ( ν , x ) = {e}^{j 2 {πℓν}_{s} x} , - M \leq ℓ \leq M , \qquad ( 22 )$$

where νₛ=2σ/(2M+1). Define aₗₘ(ν)=Rₘ(ν+lνₛ) and [symbol] (ν)=[aₗₘ(ν)]. Assume that, | [symbol] (ν)|≠0, ∀ν∈Iₛ, so that the system is invertible for any ν∈Iₛ, and that

$${w}_{m} ( x ) = \frac{1}{{ν}_{s}} {\int }_{- {ν}_{s} / 2}^{+ {ν}_{s} / 2} {W}_{m} ( ν , x ) {e}^{j 2 π ν x} d ν \qquad ( 23 )$$

can be evaluated for all m. Let d=1/νₛ. Then,

$${f}_{σ} ( x ) = {\sum }_{n = - ∞}^{∞} {\sum }_{m = - M}^{M} {y}_{m} ( n d ) {w}_{m} ( x - n d ) \qquad ( 24 )$$

is the lowpass-filtered ƒ(x), cutoff at frequency σ. The proof of the theorem is reviewed in the Appendix.

To apply the theorem, Yₖ(ν)=Fₖ(ν+kν₀) and Yₖ^{s}(ν)=Fₖ^{s}(ν+kν₀) are defined so that Eq. (4) can be written as

$${Y}_{k} ( ν ) = {R}_{k} ( ν ) F ( ν ) \qquad ( 25 )$$

with Rₖ(ν)=H(ν+kν₀) and Eq. (10) as

$${y}_{k}^{s} ( ν ) = {\sum }_{n = - ∞}^{∞} {Y}_{k} ( ν - n {ν}_{s} ) . \qquad ( 26 )$$

These equations indicate that [symbol] ₖ^{s}(x) is the sampling function associated with samples of [symbol] ₖ(x) of an LSI system having the response function rₖ(x) with input ƒ(x). Conceptually, { [symbol] ₖ^{s}(x)}_{k=-K}^{K} can be treated as available because, for ν∈Iₛ, {Fₖ^{s}(ν)}_{k=-K}^{K} can be solved from {Gₖ^{s}(ν)}_{k=-K}^{K}. If Fₖ^{s}(ν)≈0, ∀ν∈Iₛ whenever |k|≥K*+1 for some K*<K, then also can solve {Fₖ^{s}(ν)}_{k=-K*}^{K*} from {Gₖ^{s}(ν)}_{k=-K*}^{K*}. Since Fₖ^{s}(ν) is periodic, the above condition requires Fₖ(ν)≈0, ∀ν and it's previously shown that this in turn requires ƒ(x) to be (essentially) σₓ*-bandlimited (see Eq. (8)).

Therefore, according to the GST, if

$${y}_{k} ( x ) = {\int }_{- {σ}_{x , s}^{**}}^{{σ}_{x , s}^{**}} {R}_{k} ( ν ) F ( ν ) {e}^{j 2 πν x} d ν , - {K}^{* *} \leq k \leq {K}^{* *} \qquad ( 27 )$$

for some K**≤K*≤K with

$${σ}_{x , s}^{* *} = ( {K}^{* *} + 1 / 2 ) {ν}_{s} , \qquad ( 28 )$$

then ƒ_{σ}_{_{x,s}}_{^{**}}(x) can be determined from samples of yₖ(x), i.e., from samples of ƒₖ(x) because [symbol] ₖ(x)=ƒ(x) e^{-j2πkν}^{₀}^{x}. In the current treatment, no assumption about r=ν₀/νₛ has been made. Particularly, unlike in other sections, r or 1/r is not required to be a positive integer. However, later in the Appendix, the condition | [symbol] (ν)|≠0, ∀ν≠Iₛ requires

$$max \{ 0 , 1 - Γ / {K}^{* *} \} \leq r \leq 1 + Γ / {K}^{* *} , \qquad ( 29 )$$

where Γ=σ/νₛ−½. Note that 1−Γ/K**≤1≤1+Γ/K**.

A finite σ_{x,s}** in Eq. (27) may be due to the BWs of Rₖ(ν)'s or the BW of F(ν). Since H(ν)=0 for |ν|>σ, all Rₖ(ν), −K**≤k≤K** vanishes for |ν|≥σ′≡K**ν₀+σ and so Eq. (27) is valid with any σ_{x,s}**≥σ′. It is easy to check that this inequality requires ν₀<νₛ. On the other hand, σ_{x,s}**<σ′ when ν₀≥νₛ. In the latter case, to guarantee Eq. (27) ƒ(x) shall be (essentially) σ_{x,s}**-bandlimited. Of course, it also has σ_{x,s}**≤σₓ*. In some other sections, the (essential) bandlimitedness condition is needed when ν₀=pνₛ, p∈ [symbol] ⁺ but is not needed when νₛ=qν₀, q∈ [symbol] ⁺. Also, the lower bound in Eq. (29) can indefinitely approach zero and in some other sections, q is not bounded. On the other hand, both p and r are bounded from above.

### APPENDIX

### Derivation of Eq. (7)

For completeness and clarity, the Wiener-filter analytic solution of SIM in consistence with the notations used in the present disclosure is re-derived. When cₗₖ=mₖ [symbol] , from Eq. (2)

$${Y}_{k} ( ν ) ≡ {\sum }_{ℓ = 0}^{2 K} {e}^{j2 π kl / ( 2 K + 1 )} {G}_{l} ( ν ) = {m}_{k} H ( ν ) F ( ν - k {ν}_{0} ) \qquad ( 30 )$$

because Σₗ₌₀^{2K} [symbol] =δ_{kk′}. By making the substitution ν→ν+kν₀, multiplying the equation by mₖ*H*(ν+kν₀), and summing over k,

$$W ( ν ) F ( ν ) = {\sum }_{k = - K}^{K} {m}_{k}^{*} {H}^{*} ( ν + k {ν}_{0} ) {Y}_{k} ( ν + k {ν}_{0} ) , \qquad ( 31 )$$

where W(ν)=Σ_{k=-K}^{K}|mₖH(ν+kν₀)|². With real data, the RHS of this equation has an additional term due to noise that can be greatly amplified when dividing the equation by W(ν) to yield F(ν) if W(ν) is small. Eq. (7) is obtained by dividing Eq. (31) with W(ν)+α instead by using some α>0. When W(ν)>>α, W(ν)/(W(ν)+α)≈1 and Eq. (7) yields F(ν). When W(ν)<<α, it yields (W(ν)/α)F(ν).
A Necessary Conditions for | [symbol] (ν)|≠0 when ν₀=pνₛ, p∈

[symbol]

In this case, ν₀≤2σ is necessary, yielding

$$p \leq ⌊ 2 σ / {ν}_{s} ⌋ . \qquad ( 32 )$$

When p=1, the inequality is evident because ν₀=νₛ and νₛ<2σ. When p>1, it is needed for yielding | [symbol] (ν)|≠0.

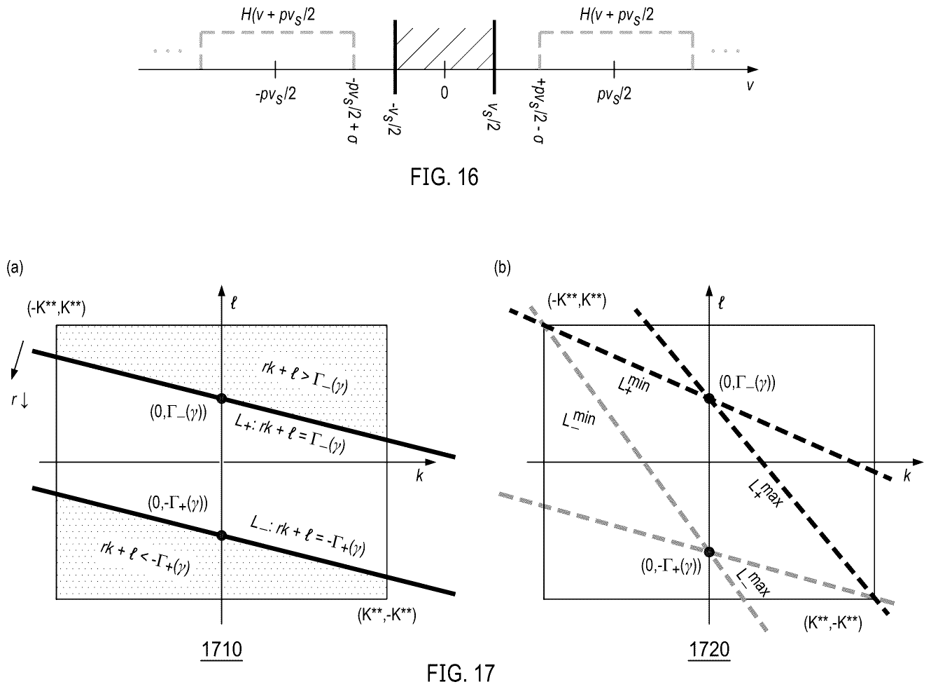

Let [symbol] (ν)= [symbol] [symbol] (ν−nνₛ). If p is even, it is easy to check that the two points on [symbol] (m=p/2) bracketing the n=0 line are (k, n)=(0, p/2) and (−1, −p/2). To [symbol] (ν), they contribute H(ν+pνₛ/2) and H(ν−pνₛ/2). If ν₀>2σ, as shown in FIG. 16, these functions do not cover the interval I_{g}=(σ₋, σ₊) where σ₋=−pνₛ/2+σ and σ₊=pνₛ/2−σ. Trivially, σ₋<0, σ₊>0, and so Iₛ∩I_{g}≠Ø. Since [symbol] (m±p)= [symbol] (m), the result is also true for all m=p/2+αp, α∈ [symbol] . If p is odd, similarly, the two points on [symbol] (m=(p−1)/2) bracketing the n=0 line are (k, n)=(0, −(p−1)/2) and (1, (p+1)/2). Then, σ₋=−(p−1)νₛ/2+σ<νₛ/2, σ₊=(p+1)νₛ/2−σ>νₛ/2, and Iₛ∩I_{g}≠Ø also. Again, using the periodicity of [symbol] (m) the result is true for all m=(p−1)/2+αp, α∈ [symbol] . Now, as replacing [symbol] (m) in [symbol] (ν) with [symbol] _{Γ}(m) to yield [symbol] (ν) can only widen I_{g}, for any p>1, there exist multiple m at the spacing p such that the sum in Eq. (16) does not cover some ν∈Iₛ. For any such ν, [symbol] (ν)=0, ∀ [symbol] . Thus, [symbol] (ν) contains columns of zeros and | [symbol] (ν)|=0.

When ν₀≤2σ, incomplete coverage of Iₛ is still possible after the range of [symbol] (m) is restricted. Consider requiring |n|≤N. Let [symbol] _{|n|≤N}(m)= [symbol] (m)∩{n:|n|≤N} and [symbol] (m)={n:(k, n)∈ [symbol] _{|n|≤N}(m)}. Given that νₛ/2<σ, Iₛ is fully covered if 0∈ [symbol] (m). Also, if n∈ [symbol] (m) where −(p−1)≤n≤−1, then sum over [symbol] _{|n|≤N}(m) includes H(ν−nνₛ) and H(ν−(n+p)νₛ) that cover the interval I=(nνₛ−σ, (n+p)νₛ+σ). Trivially, Iₛ⊆I because nνₛ−σ≤−σ and (n+p)νₛ+σ>σ. Thus, there are situations in which Iₛ is not fully covered. (a) When [symbol] (m) has only positive values and the support of H(ν−nνₛ) associated with its smallest element n* does not fully cover Iₛ. That is, n*νₛ−σ>−νₛ/2 or n >σ/νₛ−½. (b) Similarly, when [symbol] (m) has only negative values and its largest element n* satisfies n*<−(σ/νₛ−½). By symmetry, only case (a) below need to be considered. Also, it suffices to consider m=1, . . . , p−1 because [symbol] (m±p)= [symbol] (m) and 0∈ [symbol] (0).

Given an m=1, . . . , p−1, it is easy to check that the n values of the two points on [symbol] (m) bracketing n=0 are m and m−p. Therefore, case (a) occurs when m−p<−N(so that [symbol] (m) is purely positive) and m>σ/νₛ−½. Conversely, it will not occur if m−p≥−N or m≤σ/νₛ−½ when m−p≤−N−1. Now for the latter case, since m≤p−(N+1), the condition is satisfied if p−(N+1)≤σ/νₛ−½ or p=└p┘≤└N+σ/νₛ+½┘=2N. Using └2x┘≤2└x┘, Eq. (32) implies p≤2N. Therefore, case (a) will not occur under the condition given by Eq. (32).

Next, applying |k|≤K is considered. Again, by symmetry, only k≤K for m≥0 needs to be considered. Applying k≤K does not affect the positive elements of [symbol] (m). Therefore, the above analysis still applies unless the largest negative element of [symbol] (m) is eliminated. This only occurs when m>Kp. For them, after elimination by k≤K, [symbol] (m) becomes purely positive and its smallest element is n=m−Kp. When m>Kp+σ/νₛ−½, n=m−Kp>σ/νₛ−½. This is case (1) discussed above and hence Iₛ is not fully covered. Since m is an integer, the above inequality can be written as m=┌m┐≥[Kp+σ/νₛ−½]=Kp+└σ/νₛ+½┘=Kp+N=M₁. It may be concluded that Iₛ is not fully covered when m=±M₁.

Hence, under the condition given by Eq. (32), in general cₗₘ(ν)≠0, ∀ν∈Iₛ except for m=±M₁. As discussed in the text, the signal shall be (essentially) bandlimited to σ_{x,s}*=(K*+½)νₛ with K*=M₁−1.

A Necessary Condition for | [symbol] ′(ν)|≠0 when νₛ=qν₀, q∈

[symbol]

First, since νₛ≤2σ and ν₀≤νₛ by hypothesis, the condition ν₀≤2σ is also true for all q. Iₛ is fully covered by H(ν) and that adjacent H(ν−nν₀) overlap. For |m|≤K, (m, 0)∈ [symbol] _{Γ}′(m); therefore, H(ν) is included in Eq. (18) and Iₛ is fully covered. For m>K, the n values included in [symbol] _{Γ}′(m) are given by └(m−K)/q┘≤n≤N and Eq. (18) covers └(m−K)/q┘ν₀−σ≤ν≤Nν₀+σ. Clearly, Nν₀+σ>νₛ/2. Hence, Iₛ is not fully covered only when └(m−K)/q┘ν₀−σ>−νₛ/2, or └(m−K)/q>(σ/νₛ−½)q. Observing that the quantity on the left-hand side of this inequality equals 0, . . . , N for m=K+1, . . . . K+Nq and that on the RHS is positive, the smallest m* satisfying the inequality is

$${m}^{*} = K + ⌈ ( \frac{σ}{{ν}_{s}} - \frac{1}{2} ) q ⌉ q . \qquad ( 33 )$$

Again, the highest order terms given by |m|≥m* shall be eliminated by imposing a suitable (essential) bandlimited-ness assumption on the signal.

### Papoulis' Generalized Sampling Theorem

In its original form, ƒ(x) is σ-bandlimited and Eq. (24) yields ƒ(x). Papoulis' proof is below, however remains valid for the modified form of the theorem.

In Eq. (22), since [symbol] = [symbol] for any n∈ [symbol] and Rₘ(⋅) does not depend on x, Wₘ(ν, x+nd)=Wₘ(ν, x). Using this property in Eq. (23),

$${w}_{m} ( x - n d ) = \frac{1}{{ν}_{s}} {\int }_{- {ν}_{s} / 2}^{+ {ν}_{s} / 2} \{ {W}_{m} ( ν , x ) {e}^{j 2 π ν x} \} {e}^{- j 2 π n ν / {ν}_{s}} d ν , \qquad ( 34 )$$

which says that wₘ(x−nd) is the the nth FSE coefficient of Wₘ(ν, x)e^{j2πνx} with respect to ν∈Iₛ. Thus, the FSE of Wₘ(ν, x)e^{j2πνx} for ν∈Iₛ is with

$${W}_{m} ( ν , x ) {e}^{j2 π ν x} = {\sum }_{n = - ∞}^{∞} {w}_{m} ( x - n d ) {e}^{j2 π n ν / {ν}_{s} .} \qquad ( 35 )$$

Multiplying e^{j2πνx} to Eq. (22) and using Eq. (35),

$${\sum }_{m = - M}^{M} {R}_{m} ( ν + {ℓν}_{s} ) {\sum }_{n = - ∞}^{∞} {w}_{m} ( x - n d ) {e}^{j 2 π n ν d} = {e}^{j 2 π ( ν + {ℓν}_{s} ) x} , \qquad ( 36 )$$

for ν∈Iₛ. The 2M+1 equations obtained for −M≤ [symbol] ≤M can be merged to yield

$${\sum }_{m = - M}^{M} {R}_{m} ( ν ) {\sum }_{n = - ∞}^{∞} {w}_{m} ( x - n d ) {e}^{j 2 πν nd} = {e}^{j 2 πν x} \qquad ( 37 )$$

for ν∈(−σ,σ) with σ=(M+½)νₛ. Eq. (24) then follows by using Eq. (37) in the definition of ƒ_{σ}(x), given by

$${f}_{σ} ( x ) = {\int }_{- σ}^{σ} F ( ν ) {e}^{j 2 π ν x} d ν , \qquad ( 38 )$$

and using ∫_{-σ}^{σ}Rₘ(ν)F(ν)e^{j2πνnd}dν= [symbol] ₘ(nd) from Eq. (21).
A necessary Condition for | [symbol] (ν)|≠0

[symbol] (ν) cannot have any row or column of zeros. Since aₗₖ(γ)=H(γ+(rk+l)νₛ), [symbol] (γ)=0 when |γ+(rk+l)νₛ|>σ, yielding rk+ [symbol] >Γ₋(γ)≡(σ−γ)/νₛ or rk+l<−Γ+(γ)≡(σ+γ)/νₛ. Trivially, Γ_{γ}(γ)≥0 since |r|≤νₛ/2≤σ. Also, min{Γ₋(γ), Γ₊(γ)}=Γₘᵢₙ(γ)≡(σ−|γ|)/νₛ and max{Γ₋(γ), Γ₊(γ)}=Γₘₐₓ(γ)≡(σ+|γ|)/νₛ.

In 1710 of FIG. 17 showing the case Γ_{γ}(γ)<K**, the shaded areas satisfy rk+l>Γ₋(γ) or rk+l<−Γ₊(γ). A horizontal (vertical) line in these areas then yields a row (column) of zeros in [symbol] (γ). It is easy to check that, shown in 1720 of FIG. 17, rk+l=Γ₋(γ) passes through (−K**, +K**) and (K**, −K**) when r=rₘᵢₙ⁺≡1−Γ₋(γ)/K** and r=rₘₐₓ⁺≡1+Γ₋(γ)/K**, respectively. To prevent the rk+l>Γ₋(γ) area containing horizontal or vertical lines, since r≥0, rₘᵢₙ⁺(γ)≤r≤rₘₐₓ⁺(γ). If Γ₋(γ)>K**, rₘᵢₙ⁺(γ)≤0 and evidently the lower bound on r equals 0. Therefore, max{0, rₘᵢₙ⁺(γ)}≤r≤rₘₐₓ⁺(γ). Likewise, considering the rk+l<−Γ₊(γ) area yields max{0, rₘᵢₙ⁻(γ)}≤r≤rₘₐₓ⁻(γ), where rₘᵢₙ⁻(γ)=1−Γ₊(γ)/K** and rₘₐₓ⁻(γ)=1+Γ₊(γ)/K**. Combining the above conditions yields max{0, rₘᵢₙ(γ)}≤r≤rₘₐₓ(γ), where rₘᵢₙ(γ)=max{rₘᵢₙ⁻(γ), rₘᵢₙ⁺(γ)}=1−Γₘᵢₙ(γ)/K** and rₘₐₓ=min{rₘₐₓ⁻(γ), rₘₐₓ⁺(γ)}=1+Γₘᵢₙ(γ)/K**. For this inequality to hold for all |γ|≤νₛ/2, it must to have max{0, rₘᵢₙ}≤r≤rₘₐₓ, where rₘᵢₙ=max{rₘᵢₙ(γ)}=1−Γ/K** and rₘₐₓ=min{rₘₐₓ(γ)}=1+Γ/K** with Γ=σ/νₛ−½.

### Super-Resolution Embodiments: Super-Resolution PET

The present disclosure describes various embodiments, wherein, for extending the theory of structured illumination microscopy (SIM), a rotating modulator is used in front of a stationary PET detector ring for increasing the resolution of clinical PET systems. Simulation data shows that 0.9 mm sources can be resolved when using noise-free data produced by a PET system consisting of 576 4.2 mm-width crystals. In practice, data noise may limit resolution recovery. Results with noisy data are obtained when using the ordered-subset expectation-maximization algorithm, which is susceptible to noise. Hence, the method can be practical with optimization of the design of the modulator and the reconstruction method.

### Introduction

Positron emission tomography (PET) is an in vivo molecular imaging modality having demonstrated clinical value. Presently, the resolution of clinical PET imaging is about 4-6 mm. While suitable for whole-body imaging, this resolution is insufficient for resolving structures in some organs such as the brain. Improving the resolution in clinical PET imaging therefore has been a topic of interest because dedicated high-resolution systems for specific organs are not readily available in practice. Several approaches for compensating the resolution-limiting effects in PET have been proposed, including, as clinical PET resolution is fundamentally limited by the crystal size, the idea of using collimators to effectively decrease the crystal size in conjunction with collimator movements to provide suitable sampling. In practice, data noise will limit resolution recovery. But the latter approach has been shown to yield better visualization and quantification for small structures even though the coincidence detection efficiency (CDE) is reduced by 75% due to collimation.

In microscopy, various super-resolution (SR) techniques that can surpass the resolution limit due to diffraction of light have been developed. The essential idea in the method of structured illumination microscopy (SIM) is to modulate the image field so that its high-frequency content above the bandwidth of the device, hence undetectable, are encoded into lower-frequency, detectable signals. The modulation pattern is moved to a number of positions for producing multiple resolution-limited images that however contain encoded high-frequency content of the image field under examination. Then, an SR image is produced from these images by computationally restoring signals to correct frequencies. This work is based on: (1) the observation that the modulation step in SIM can be implemented in PET by use of a rotating, partially attenuating ring in front of the PET detectors, and (2) the hypothesis that the linear, shift-invariant SIM theory can be successful when extended to shift-variant PET systems. There is no theoretical limit to the highest recoverable frequency with the so-called nonlinear SIM. In practice, it can be limited by the characteristics of PET imaging physics and will be limited by data noise. The objective of this work is to show a proof-of-concept for this SIM inspired approach by using simulated noise-free data and also to examine whether it can be successful for noisy data.

### Method Steps in Various Embodiments

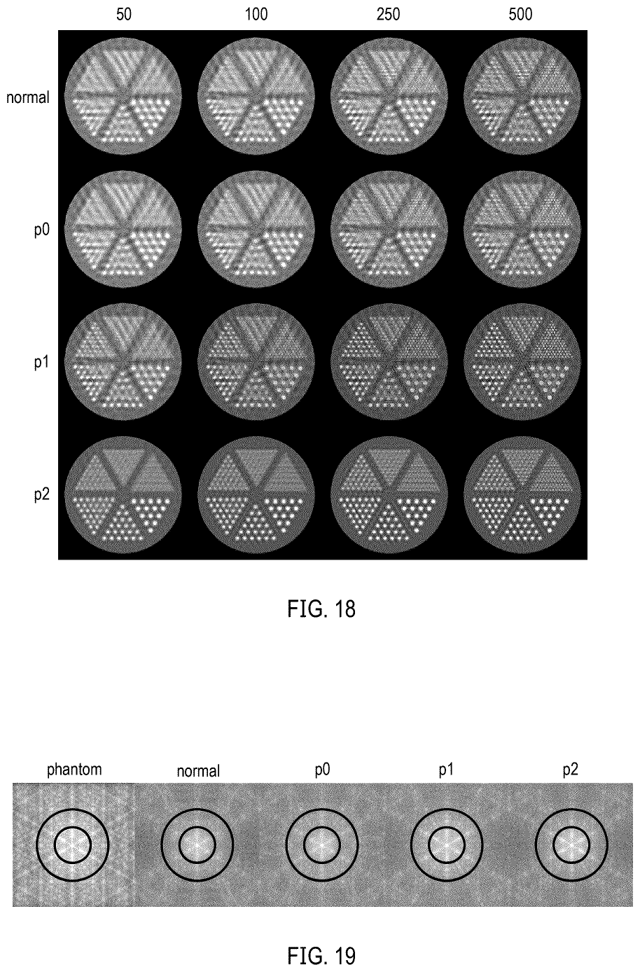

Based on the design of a developed PET system, a 77.0 cm-diameter PET ring contained 576 crystals whose width d was 4.2 mm (comparable with most existing clinical systems) is considered. The modulator provided n segments (n=3 shown) of different attenuation for annihilation photons before they enter a crystal. In simulation, a crystal is divided into m subcrystals of equal width and defined a virtual line of response (LOR) by connecting the centers of two subcrystals. Raytracing was performed, using the Siddon method, to compute the activity raysum along all virtual LORs for a numerical phantom. Next, the raysum was multiplied by the product of the attenuation in front of its two subcrystals belonging to a crystal pair. Then, noise-free data for a given crystal pair was obtained by summing the attenuated raysums at its m² virtual LORs. Using a larger m could increase the modeling accuracy but require more computation. Some implementations used m=6. FIG. 18 shows reconstruction images with different sampling modes and iterations. From the top to bottom rows are normal, p0, p1, p2 mode. From left to right columns, the number of iterations is 50, 100, 250, and 500.

By using different n and different attenuation for different segments, various modulation patterns in front of the crystals can be created. Three patterns were examined. The M0.5 modulator used n=6 and its period was d/2. The M1 and M2 modulators used n=3 and their periods was d and 2d respectively. FIG. 1(b) shows an example of the modulation created for coincidence photons in a projection view. With the M0.5, M1, and M2 modulators, the CDE were 0.4286, 0.4287, 0.4517 times the CDE when no modulator was used, denoted as M−. FIG. 1(c) shows the phantom used had six groups of hot sources of different diameters, including 0.9, 1.2, 1.5, 1.8, 2.1, and 2.4 mm, on top of a 69.0 mm diameter disc. The hot-source to background activity ratio was 4:1. The phantom was an 256×256 array of 0.3×0.3 mm² pixels.

For a given modulator, three noise-free datasets were generated by rotating the modulator by ⅓ the angular spacing of a crystal. The three datasets were then juxtaposed to a form a larger one, which could be related to the unknown image by a matrix equation as in conventional PET but now the system matrix included the effects of the modulator at the rotational positions used. An ordered-subset expectation-maximization (OSEM) algorithm was implemented to solve the matrix equation, for a 256×256 image array consisting of 0.3×0.3 mm² pixels. The system matrix was computed on-the-fly during iteration following the above procedure for generating noise-free data.

For noisy data, Poisson noise was added. The noise level was specified in terms of the expected total number of counts N for the case when no modulator was used. When modulator was used, N was automatically scaled in accordance with reduction in CDE it incurred so that the noisy-data results could be compared under the same scan-duration condition.

### Exemplary Results of Various Embodiments

In some implementations, images obtained from noise-free data with using modulators to those obtained without (M−), by 50 and 500 OSEM iterations. Subjectively and generally, more iterations leads to higher resolution, M0.5 and M− images are similar, and M2 images have the best quality. At 50 iterations, the M2 mage can resolve 1.5 mm sources. In contrast, the M− image has streaks and cannot confidently resolve sources smaller 2.1 mm. At 500 iterations, while showing improved resolution, streaks persist in the M− image. Both M1 and M2 images can resolve 0.9 mm sources, with the latter being subjectively better.

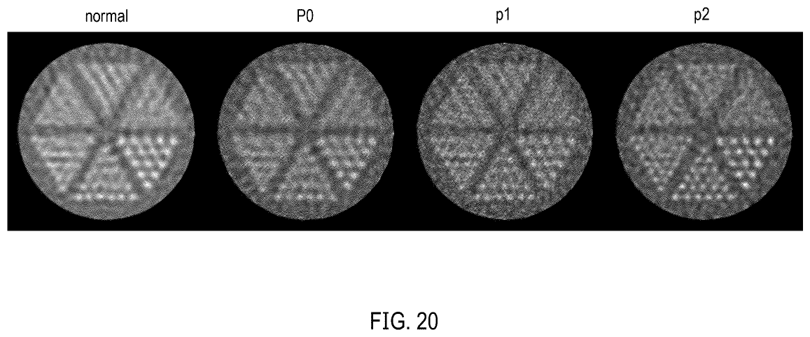

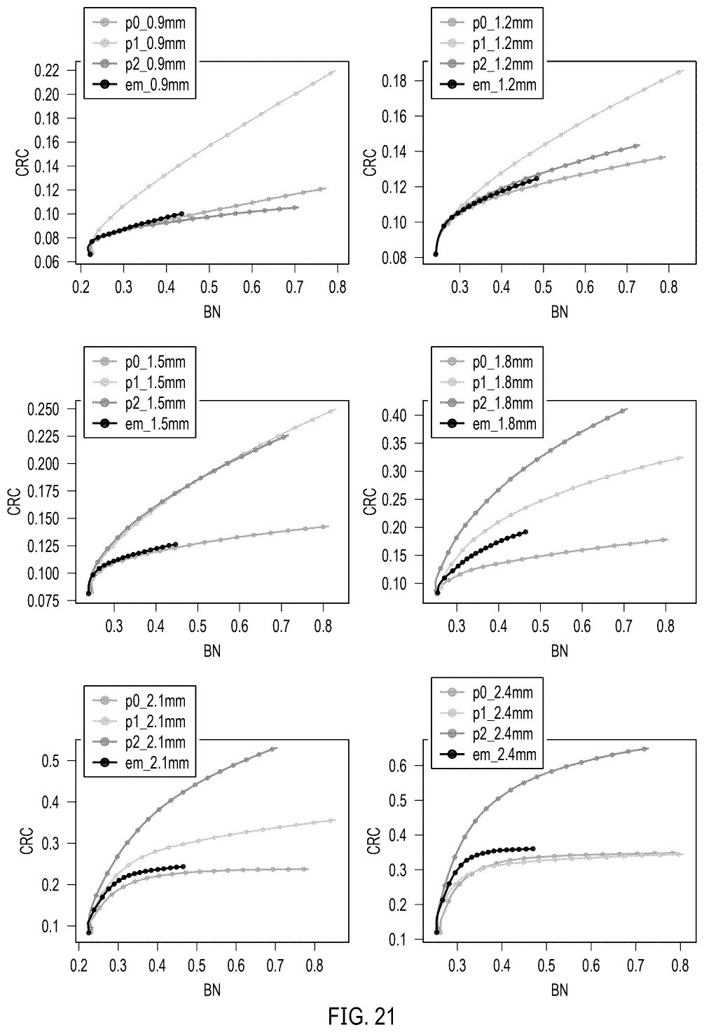

FIG. 19 shows spectrum of phantom and four sampling mode in 500 iterations. The inner black circle is theoretical cutoff frequency and the outer one is double of it. FIG. 20 shows reconstruction images of four different sampling modes at the same scan time relative to normal sampling mode. FIG. 21 shows CRC-BN curves at different rod diameters from 0.9 mm to 2.4 mm (top left to bottom right).

In some implementations, images obtained from an N=8×10⁶ (8M) noisy data (note that number was scaled by the reduced CDE when modulator was used) after 20 OSEM iterations. In some implementations, the contrast-recovery-coefficient (CRC) versus background noise (BN) curves, calculated for each size group, obtained from such 8M images as the iteration number increases. Consistently with the subjective quality, in comparison with the M− result, at the same BN the M2 result has significantly higher CRC (comparable CRC) for sources larger (smaller) than 1.5 mm. On the other hand, M0.5 yields lower CRC than M− for all sources while M1 yields higher CRC than M− except for the largest source. More studies are needed to understand these behaviors.

In some other embodiments, a computer-readable medium comprising instructions which, when executed by a computer, cause the computer to carry out the above methods. The computer-readable medium may be referred as non-transitory computer-readable media (CRM) that stores data for extended periods such as a flash drive or compact disk (CD), or for short periods in the presence of power such as a memory device or random access memory (RAM). In some embodiments, computer-readable instructions may be included in a software, which is embodied in one or more tangible, non-transitory, computer-readable media. Such non-transitory computer-readable media can be media associated with user-accessible mass storage as well as certain short-duration storage that are of non-transitory nature, such as internal mass storage or ROM. The software implementing various embodiments of the present disclosure can be stored in such devices and executed by a processor (or processing circuitry). A computer-readable medium can include one or more memory devices or chips, according to particular needs. The software can cause the processor (including CPU, GPU, FPGA, and the like) to execute particular processes or particular parts of particular processes described herein, including defining data structures stored in RAM and modifying such data structures according to the processes defined by the software.

Reference throughout this specification to features, advantages, or similar language does not imply that all of the features and advantages that may be realized with the present solution should be or are included in any single implementation thereof. Rather, language referring to the features and advantages is understood to mean that a specific feature, advantage, or characteristic described in connection with an embodiment is included in at least one embodiment of the present solution. Thus, discussions of the features and advantages, and similar language, throughout the specification may, but do not necessarily, refer to the same embodiment.

Furthermore, the described features, advantages and characteristics of the present solution may be combined in any suitable manner in one or more embodiments. One of ordinary skill in the relevant art will recognize, in light of the description herein, that the present solution can be practiced without one or more of the specific features or advantages of a particular embodiment. In other instances, additional features and advantages may be recognized in certain embodiments that may not be present in all embodiments of the present solution.

What is claimed:

1. An apparatus for acquiring a super-resolution image of a subject, the apparatus comprising:
a periodic pattern and a detector, wherein:
the periodic pattern is configured to move to modify an original image field before the original image field propagates onto the detector of an imaging system, the original image field originating from a subject and comprising information of the subject, and
the detector is configured to detect the modified original image field to acquire a plurality of detected images; and
a memory storing instructions and a processor in communication with the memory, wherein, when the processor executes the instructions, the processor is configured to cause the apparatus to reconstruct a super-resolution image based on the plurality of detected images, wherein an imaging resolution of the super-resolution image is better than an intrinsic resolution of the detector.

2. The apparatus according to claim 1, wherein:
the reconstructed super-resolution image has a higher spatial frequency than a spatial frequency of the imaging system.

3. The apparatus according to claim 1, wherein:
a rotatable mask comprises the periodic pattern;
the rotatable mask is configured to constantly rotate; and
the detector is configured to constantly detect the modified original image field to acquire the plurality of detected images, wherein each of the plurality of detected images is indexed with information corresponding to a position of the periodic pattern when the detected image is acquired.

4. The apparatus according to claim 1, wherein:
a rotatable mask comprises the periodic pattern;
the rotatable mask is configured to rotate in a step-by-step manner; and
the detector is configured to detect the modified original image field to acquire the plurality of detected images in a step-and-shoot manner corresponding to the rotatable mark, wherein each of the plurality of detected images is indexed with information corresponding to a position of the periodic pattern when the detected image is acquired.

5. The apparatus according to claim 1, wherein:
the detector comprises at least one of the following: a charge coupled device (CCD) for detecting optical field, a positron emission tomography (PET) detector for detecting radiation field, or a single photon emission computed tomography (SPECT) detector for detecting radiation field.

6. The apparatus according to claim 1, wherein:
the periodic pattern comprises one of the following: a structured absorptive pattern, or a structured sensitivity pattern.

7. The apparatus according to claim 1, wherein:
the periodic pattern comprises higher order harmonics.

8. The apparatus according to claim 1, wherein:
the periodic pattern has a spatial frequency higher than a spatial frequency of the imaging system.

9. The apparatus according to claim 1, wherein:
the periodic pattern comprises one or more periodic absorptive patterns.

10. The apparatus according to claim 1, wherein:
the periodic pattern comprises two orthogonal periodic absorptive patterns.

11. The apparatus according to claim 1, wherein:
the periodic pattern comprises a periodic multi-level absorption or sensitivity pattern.

12. A method for acquiring a super-resolution image of a subject, the method comprising:
moving a periodic pattern to modify an original image field before the original image field propagates onto a detector of an imaging system, the original image field originating from a subject and comprising information of the subject;
detecting the modified original image field with the detector to acquire a plurality of detected images; and
reconstructing a super-resolution image based on the plurality of detected images, wherein an imaging resolution of the super-resolution image is better than an intrinsic resolution of the detector.

13. The method according to claim 12, wherein:
the reconstructed super-resolution image has a higher spatial frequency than a spatial frequency of the imaging system.

14. The method according to claim 12, wherein:
the moving the periodic pattern to modify the original image field comprises:
constantly rotating a rotatable mask comprising the periodic pattern; and
the detecting the modified original image field with the detector to acquire the plurality of detected images comprises:
constantly detecting the modified original image field with the detector to acquire the plurality of detected images, wherein each of the plurality of detected images is indexed with information corresponding to a position of the periodic pattern when the detected image is acquired.

15. The method according to claim 12, wherein:
the moving the periodic pattern to modify the original image field comprises:
rotating a rotatable mask comprising the periodic pattern in a step-by-step manner; and
the detecting the modified original image field with the detector to acquire the plurality of detected images comprises:
detecting the modified original image field with the detector to acquire the plurality of detected images in a step-and-shoot manner corresponding to the rotatable mark, wherein each of the plurality of detected images is indexed with information corresponding to a position of the periodic pattern when the detected image is acquired.

16. The method according to claim 12, wherein:
the detector comprises at least one of the following: a charge coupled device (CCD) for detecting optical field, a positron emission tomography (PET) detector for detecting radiation field, or a single photon emission computed tomography (SPECT) detector for detecting radiation field.

17. An apparatus for acquiring a super-resolution image of a subject, the apparatus comprising:
a detector of an image system, wherein:
a modulation of the detector is configured to cause modulation of radiation beams, the radiation beams originating from a subject and comprising information of the subject, and
the detector is configured to receive the radiation beam to acquire a plurality of detected images; and
a memory storing instructions and a processor in communication with the memory, wherein, when the processor executes the instructions, the processor is configured to cause the apparatus to reconstruct a super-resolution image based on the plurality of detected images, wherein an imaging resolution of the super-resolution image is better than an intrinsic resolution of the detector.

18. The apparatus according to claim 17, wherein:
the reconstructed super-resolution image has a higher spatial frequency than a spatial frequency of the imaging system.

19. The apparatus according to claim 17, wherein:
the modulation of the detector is at a sensor level, wherein the detector comprises a plurality of position sensitive sensors, whose output is amplified with a different gain depending on a corresponding detection position with a sensitivity profile being modified electronically.

20. The apparatus according to claim 17, wherein:
the plurality of position sensitive sensors comprises at least one of the following: a position sensitive photo-multiplier tube (PMT), a position sensitive avalanche photo-diode (APD), a position sensitive Silicon photo-multiplier (SiPM).
